SEQUENCE LISTING (9 sequences)

SEQ ID NO: 1 - protein, 9 aa, Artificial Sequence
  viral chimeric antigen
NLVPMVATV

SEQ ID NO: 2 - protein, 10 aa, Artificial Sequence
  viral chimeric antigen
LLDFVRFMGV

SEQ ID NO: 3 - protein, 9 aa, Artificial Sequence
  viral chimeric antigen
GILGFVFTL

SEQ ID NO: 4 - DNA, 2490 bp, Artificial Sequence
  EBNA3C-pp65 coding strand
atggaaagcttcgagggccagggcgacagcaggcagagccccgacaacgagcggggcgacaacgtgcagaccaccggcgagcacgaccaggaccctggccctggccctcctagcagcggcgccagcgagaggctggtgcccgaggaaagctacagccgggaccagcagccctggggccagagcaggggcgacgagaaccggggctggatgcagcggatccggcggaggcggcggagaagagccgccctgagcggccacctgctggacaccgaggacaacgtgcccccctggctgcccccccacgacatcaccccctacaccgcccggaacatccgggacgccgcctgcagggccgtgaagcagagccacctgcaggccctgagcaacatggaaagccggggcaggcggtgccccgagatgatcagcgtgctgggccccatctccggccacgtgctgaaggccgtgttcagcagaggcgacacccccgtgctgccccacgagacccggctgctgcagaccggcatccacgtgagagtgagccagcccagcctgatcctggtgtcccagtacacccccgactccaccccctgccaccgcggcgacaaccagctgcaggtgcagcacacctacttcaccggcagcgaggtggagaacgtgagcgtgaacgtgcacaaccccaccggcagaagcatctgccccagccaggaacccatgagcatctacgtgtacgccctgcccctgaagatgctgaacatccccagcatcaacgtgcaccactacccctctgccgccgagcggaagcaccggcacctgcccgtggccgacgccgtgatccacgccagcggcaagcagatgtggcaggcccggctgaccgtgagcggcctggcctggacccggcagcagaaccagtggaaagaacccgacgtgtactacaccagcgccttcgtgttccccaccaaggacgtggccctgcggcacgtggtgtgcgcccacgagctggtgtgcagcatggaaaacacccgggccaccaagatgcaggtgatcggcgaccagtacgtgaaggtgtacctggaatccttctgcgaggacgtgcctagcggcaagctgttcatgcacgtgaccctgggcagcgacgtggaagaggacctgaccatgacccggaacccccagcccttcatgcggccccacgagcggaacggcttcaccgtgctgtgccccaagaacatgatcatcaagcccggcaagatcagccacatcatgctggacgtggccttcaccagccacgagcacttcggcctgctgtgccctaagtccatccccggcctgagcatcagcggcaatctgctgatgaacggccagcagatcttcctggaagtgcaggccatccgggagaccgtggagctgcggcagtacgaccccgtggccgccctgttcttcttcgacatcgacctgctgctgcagcggggaccccagtacagcgagcaccccaccttcacctcccagtaccggatccagggcaagctggaataccggcacacctgggaccggcacgacgaaggcgccgcccagggcgacgacgacgtgtggaccagcggcagcgacagcgacgaggaactggtgaccaccgagagaaagacccccagggtgaccggcggaggggccatggccggagccagcaccagcgccggcaggtctgccagcagcgccaccgcctgcacagccggcgtgatgacccggggcagactgaaggccgagtctaccgtggctcctgaagaggacaccgacgaggacagcgacaacgagatccacaaccccgccgtgttcacctggcccccttggcaggccggcatcctggcccggaacctggtgcccatggtggccaccgtgcagggccagaacctgaagtaccaggaatttttctgggacgccaacgacatctaccggatcttcgccgagctggaaggagtgtggcagccagctgcccagcccaagcggcggagacaccggcaggacgccctgcctggcccctgcatcgccagcacccccaagaagcacagaggccccctggtggccgagggcggcgtggggtggaggcactggctgctgaccagcccctcccagagctggcccatgggctaccggacagctacactgagaactctgacccccgtgcccaacagagtgggcgccgacagcatcatgctgaccgccaccttcggctgccagaacgccgccagaaccctgaacaccttctccgccaccgtgtggacccctccccacgccggaccccgggagcaggaaagatacgcccgggaggccgaagtgcggttcctgcggggcaagtggcagcggcggtacaggcggatctacgacctgatcgagctgtgcggcagcctgcaccacatctggcagaacctgctgcagacagaggaaaacctgctggacttcgtgcggttcatgggcgtgatgagcagctgcaacaacccctaa

SEQ ID NO: 5 - DNA, 2490 bp, Artificial Sequence
  EBNA3C/pp65/EBNA3C non-coding strand
tacctttcgaagctcccggtcccgctgtcgtccgtctcggggctgttgctcgccccgctgttgcacgtctggtggccgctcgtgctggtcctgggaccgggaccgggaggatcgtcgccgcggtcgctctccgaccacgggctcctttcgatgtcggccctggtcgtcgggaccccggtctcgtccccgctgctcttggccccgacctacgtcgcctaggccgcctccgccgcctcttctcggcgggactcgccggtggacgacctgtggctcctgttgcacggggggaccgacgggggggtgctgtagtgggggatgtggcgggccttgtaggccctgcggcggacgtcccggcacttcgtctcggtggacgtccgggactcgttgtacctttcggccccgtccgccacggggctctactagtcgcacgacccggggtagaggccggtgcacgacttccggcacaagtcgtctccgctgtgggggcacgacggggtgctctgggccgacgacgtctggccgtaggtgcactctcactcggtcgggtcggactaggaccacagggtcatgtgggggctgaggtgggggacggtggcgccgctgttggtcgacgtccacgtcgtgtggatgaagtggccgtcgctccacctcttgcactcgcacttgcacgtgttggggtggccgtcttcgtagacggggtcggtccttgggtactcgtagatgcacatgcgggacggggacttctacgacttgtaggggtcgtagttgcacgtggtgatggggagacggcggctcgccttcgtggccgtggacgggcaccggctgcggcactaggtgcggtcgccgttcgtctacaccgtccgggccgactggcactcgccggaccggacctgggccgtcgtcttggtcacctttcttgggctgcacatgatgtggtcgcggaagcacaaggggtggttcctgcaccgggacgccgtgcaccacacgcgggtgctcgaccacacgtcgtaccttttgtgggcccggtggttctacgtccactagccgctggtcatgcacttccacatggaccttaggaagacgctcctgcacggatcgccgttcgacaagtacgtgcactgggacccgtcgctgcaccttctcctggactggtactgggccttgggggtcgggaagtacgccggggtgctcgccttgccgaagtggcacgacacggggttcttgtactagtagttcgggccgttctagtcggtgtagtacgacctgcaccggaagtggtcggtgctcgtgaagccggacgacacgggattcaggtaggggccggactcgtagtcgccgttagacgactacttgccggtcgtctagaaggaccttcacgtccggtaggccctctggcacctcgacgccgtcatgctggggcaccggcgggacaagaagaagctgtagctggacgacgacgtcgcccctggggtcatgtcgctcgtggggtggaagtggagggtcatggcctaggtcccgttcgaccttatggccgtgtggaccctggccgtgctgcttccgcggcgggtcccgctgctgctgcacacctggtcgccgtcgctgtcgctgctccttgaccactggtggctctctttctgggggtcccactggccgcctccccggtaccggcctcggtcgtggtcgcggccgtccagacggtcgtcgcggtggcggacgtgtcggccgcactactgggccccgtctgacttccggctcagatggcaccgaggacttctcctgtggctgctcctgtcgctgttgctctaggtgttggggcggcacaagtggaccgggggaaccgtccggccgtaggaccgggccttggaccacgggtaccaccggtggcacgtcccggtcttggacttcatggtccttaaaaagaccctgcggttgctgtagatggcctagaagcggctcgaccttcctcacaccgtcggtcgacgggtcgggttcgccgcctctgtggccgtcctgcgggacggaccggggacgtagcggtcgtgggggttcttcgtgtctccgggggaccaccggctcccgccgcaccccacctccgtgaccgacgactggtcggggagggtctcgaccgggtacccgatggcctgtcgatgtgactcttgagactgggggcacgggttgtctcacccgcggctgtcgtagtacgactggcggtggaagccgacggtcttgcggcggtcttgggacttgtggaagaggcggtggcacacctggggaggggtgcggcctggggccctcgtcctttctatgcgggccctccggcttcacgccaaggacgccccgttcaccgtcgccgccatgtccgcctagatgctggactagctcgacacgccgtcggacgtggtgtagaccgtcttggacgacgtctgtctccttttggacgacctgaagcacgccaagtacccgcactactcgtcgacgttgttggggatt

SEQ ID NO: 6 - protein, 829 aa, Artificial Sequence
  EBNA3C/pp65/EBNA3C viral chimeric antigen
MESFEGQGDSRQSPDNERGDNVQTTGEHDQDPGPGPPSSGASERLVPEESYSRDQQPWGQSRGDENRGWMQRIRRRRRRRAALSGHLLDTEDNVPPWLPPHDITPYTARNIRDAACRAVKQSHLQALSNMESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRGPLVAEGGVGWRHWLLTSPSQSWPMGYRTATLRTLTPVPNRVGADSIMLTATFGCQNAARTLNTFSATVWTPPHAGPREQERYAREAEVRFLRGKWQRRYRRIYDLIELCGSLHHIWQNLLQTEENLLDFVRFMGVMSSCNNP

SEQ ID NO: 7 - DNA, 2706 bp, Artificial Sequence
  EBNA3C-pp65-MP1
atggaaagcttcgagggccagggcgacagcaggcagagccccgacaacgaaacgtgcagaccaccggcgagcacgaccaggaccctggccctggccctccgccagcgagaggctggtgcccgaggaaagctacagccgggaccagcagccagcaggggcgacgagaaccggggctggatgcagcggatccggcggaggcggccgccctgagcggccacctgctggacaccgaggacaacgtgcccccctgcacgacatcaccccctacaccgcccggaacatccgggacgccgcctgcagcagagccacctgcaggccctgagcaacatggaaagccggggcaggcggtgatcagcgtgctgggccccatctccggccacgtgctgaaggccgtgttcagacccccgtgctgccccacgagacccggctgctgcagaccggcatccacgtcagcccagcctgatcctggtgtcccagtacacccccgactccaccccctggacaaccagctgcaggtgcagcacacctacttcaccggcagcgaggtggagtgaacgtgcacaaccccaccggcagaagcatctgccccagccaggaacctacgtgtacgccctgcccctgaagatgctgaacatccccagcatcaacgtccctctgccgccgagcggaagcaccggcacctgcccgtggccgacgccgtagcggcaagcagatgtggcaggcccggctgaccgtgagcggcctggcctgcagaaccagtggaaagaacccgacgtgtactacaccagcgccttcgtgttgacgtggccctgcggcacgtggtgtgcgcccacgagctggtgtgcagcatcgggccaccaagatgcaggtgatcggcgaccagtacgtgaaggtgtaccttgcgaggacgtgcctagcggcaagctgttcatgcacgtgaccctgggcaggaggacctgaccatgacccggaacccccagcccttcatgcggccccacgattcaccgtgctgtgccccaagaacatgatcatcaagcccggcaagatcagctggacgtggccttcaccagccacgagcacttcggcctgctgtgccctaaggcctgagcatcagcggcaatctgctgatgaacggccagcagatcttcctgccatccgggagaccgtggagctgcggcagtacgaccccgtggccgccctgacatcgacctgctgctgcagcggggaccccagtacagcgagcaccccaccagtaccggatccagggcaagctggaataccggcacacctgggaccggcagccgcccagggcgacgacgacgtgtggaccagcggcagcgacagcgacgaaccaccgagagaaagacccccagggtgaccggcggaggggccatggccggagcgccggcaggtctgccagcagcgccaccgcctgcacagccggcgtgatagactgaaggccgagtctaccgtggctcctgaagaggacaccgacgaggagagatccacaaccccgccgtgttcacctggcccccttggcaggccggcataacctggtgcccatggtggccaccgtgcagggccagaacctgaagtaccatgggacgccaacgacatctaccggatcttcgccgagctggaaggagtgtggcccagcccaagcggcggagacaccggcaggacgccctgcctggcccctgacccccaagaagcacagaggccccctggtggccgagggcggcgtggggtgctgctgaccagcccctcccagagctggcccatgggctaccggacagctacctgacccccgtgcccaacagagtgggcgccgacagcatcatgctgaccgctgccagaacgccgccagaaccctgaacaccttctccgccaccgtgtggacgccggaccccgggagcaggaaagatacgcccgggaggccgaagtgcggttaagtggcagcggcggtacaggcggatctacgacctgatcgagctgtgcggcacatctggcagaacctgctgcagacagaggaaaacctgctggacttcgtggcgtgatgagcagctgcaacaaccccatgagcctgctgacagaggtggactgtccatcgtgcccagcggccctctgaaagccgagatcgcccagcggctttcgccggcaagaacaccgatctggaagtgctgatggaatggctgaaaacctgagccccctgaccaagggcatcctgggcttcgtgttcaccctgaccgtcggggcctgcagcggaggcggttcgtgcagaacgccctgaacggcaacggaacatggacaaggccgtgaaactgtaccggaagctgaagcgggagatcacgccaaagagatcgccctgagctacagcgccggagccctggccagctgcattacaaccggatgggcgccgtgaccacagaggtggcctttggcctggtgtggagcagatcgccgacagccagcacagatcccacaggcagatggtgaccacctgatccggcacgagaacaggatggtgctggcctccaccaccgccaaggcatggccggcagcagcgagcaggccgccgaagccatggacatcgccagccaatggtgcaggccatgcggaccatcggcacccaccccagcagctctgccgggacctgctggaaaatctgcaggcctaccagaaacggatgggcgtgcagataagtga

SEQ ID NO: 8 - DNA, 3246 bp, Artificial Sequence
  EBNA3C-pp65-MP1
tacctttcgaagctcccggtcccgctgtcgtccgtctcggggctgttgctcgccccgctgttgcacgtctggtggccgctcgtgctggtcctgggaccgggaccgggaggatcgtcgccgcggtcgctctccgaccacgggctcctttcgatgtcggccctggtcgtcgggaccccggtctcgtccccgctgctcttggccccgacctacgtcgcctaggccgcctccgccgcctcttctcggcgggactcgccggtggacgacctgtggctcctgttgcacggggggaccgacgggggggtgctgtagtgggggatgtggcgggccttgtaggccctgcggcggacgtcccggcacttcgtctcggtggacgtccgggactcgttgtacctttcggccccgtccgccacggggctctactagtcgcacgacccggggtagaggccggtgcacgacttccggcacaagtcgtctccgctgtgggggcacgacggggtgctctgggccgacgacgtctggccgtaggtgcactctcactcggtcgggtcggactaggaccacagggtcatgtgggggctgaggtgggggacggtggcgccgctgttggtcgacgtccacgtcgtgtggatgaagtggccgtcgctccacctcttgcactcgcacttgcacgtgttggggtggccgtcttcgtagacggggtcggtccttgggtactcgtagatgcacatgcgggacggggacttctacgacttgtaggggtcgtagttgcacgtggtgatggggagacggcggctcgccttcgtggccgtggacgggcaccggctgcggcactaggtgcggtcgccgttcgtctacaccgtccgggccgactggcactcgccggaccggacctgggccgtcgtcttggtcacctttcttgggctgcacatgatgtggtcgcggaagcacaaggggtggttcctgcaccgggacgccgtgcaccacacgcgggtgctcgaccacacgtcgtaccttttgtgggcccggtggttctacgtccactagccgctggtcatgcacttccacatggaccttaggaagacgctcctgcacggatcgccgttcgacaagtacgtgcactgggacccgtcgctgcaccttctcctggactggtactgggccttgggggtcgggaagtacgccggggtgctcgccttgccgaagtggcacgacacggggttcttgtactagtagttcgggccgttctagtcggtgtagtacgacctgcaccggaagtggtcggtgctcgtgaagccggacgacacgggattcaggtaggggccggactcgtagtcgccgttagacgactacttgccggtcgtctagaaggaccttcacgtccggtaggccctctggcacctcgacgccgtcatgctggggcaccggcgggacaagaagaagctgtagctggacgacgacgtcgcccctggggtcatgtcgctcgtggggtggaagtggagggtcatggcctaggtcccgttcgaccttatggccgtgtggaccctggccgtgctgcttccgcggcgggtcccgctgctgctgcacacctggtcgccgtcgctgtcgctgctccttgaccactggtggctctctttctgggggtcccactggccgcctccccggtaccggcctcggtcgtggtcgcggccgtccagacggtcgtcgcggtggcggacgtgtcggccgcactactgggccccgtctgacttccggctcagatggcaccgaggacttctcctgtggctgctcctgtcgctgttgctctaggtgttggggcggcacaagtggaccgggggaaccgtccggccgtaggaccgggccttggaccacgggtaccaccggtggcacgtcccggtcttggacttcatggtccttaaaaagaccctgcggttgctgtagatggcctagaagcggctcgaccttcctcacaccgtcggtcgacgggtcgggttcgccgcctctgtggccgtcctgcgggacggaccggggacgtagcggtcgtgggggttcttcgtgtctccgggggaccaccggctcccgccgcaccccacctccgtgaccgacgactggtcggggagggtctcgaccgggtacccgatggcctgtcgatgtgactcttgagactgggggcacgggttgtctcacccgcggctgtcgtagtacgactggcggtggaagccgacggtcttgcggcggtcttgggacttgtggaagaggcggtggcacacctggggaggggtgcggcctggggccctcgtcctttctatgcgggccctccggcttcacgccaaggacgccccgttcaccgtcgccgccatgtccgcctagatgctggactagctcgacacgccgtcggacgtggtgtagaccgtcttggacgacgtctgtctccttttggacgacctgaagcacgccaagtacccgcactactcgtcgacgttgttggggtactcggacgactgtctccacctctggatgcacgacaggtagcacgggtcgccgggagactttcggctctagcgggtcgccgaccttctacacaagcggccgttcttgtggctagaccttcacgactaccttaccgacttttgggccgggtaggactcgggggactggttcccgtaggacccgaagcacaagtgggactggcacgggaggctcgccccggacgtcgcctccgccaagcacgtcttgcgggacttgccgttgccgctggggttgttgtacctgttccggcactttgacatggccttcgacttcgccctctagtggaaggtgccgcggtttctctagcgggactcgatgtcgcggcctcgggaccggtcgacgtacccggactagatgttggcctacccgcggcactggtgtctccaccggaaaccggaccacacgcggtggacgctcgtctagcggctgtcggtcgtgtctagggtgtccgtctaccactggtgttggttgggggactaggccgtgctcttgtcctaccacgaccggaggtggtggcggttccggtaccttgtctaccggccgtcgtcgctcgtccggcggcttcggtacctgtagcggtcggtccggtctgtctaccacgtccggtacgcctggtagccgtgggtggggtcgtcgagacggccggacttcctgctggacgaccttttagacgtccggatggtctttgcctacccgcacgtctacgtcgccaagttcact

SEQ ID NO: 9 - protein, 1081 aa, Artificial Sequence
  EBNA3C-pp65{circumflex over ())}{circumflex over (})}k-MP1
MESFEGQGDSRQSPDNERGDNVQTTGEHDQDPGPGPPSSGASERLVPEESYSRDQQPWGQSRGDENRGWMQRIRRRRRRRAALSGHLLDTEDNVPPWLPPHDITPYTARNIRDAACRAVKQSHLQALSNMESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRGPLVAEGGVGWRHWLLTSPSQSWPMGYRTATLRTLTPVPNRVGADSIMLTATFGCQNAARTLNTFSATVWTPPHAGPREQERYAREAEVRFLRGKWQRRYRRIYDLIELCGSLHHIWQNLLQTEENLLDFVRFMGVMSSCNNPMSLLTEVETYVLSIVPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVTTTNPLIRHENRMVLASTTAKAMEQMAGSSEQAAEAMDIASQARQMVQAMRTIGTHPSSSAGLKDDLLENLQAYQKRMGVQMQRFK

(19) United States
(12) Patent Application Publication
(10) Pub. No.: US 2009/0324630 A1
(43) Pub. Date: Dec. 31, 2009

(54) FUSION MULTIVIRAL CHIMERIC ANTIGEN

(76) Inventor: Michael C. Jensen, Sierra Madre, CA (US)

Correspondence Address: PERKINS COIE LLP, POST OFFICE BOX 1208, SEATTLE, WA, 98111-1208, US

(21) Appl. No.: 12/427,697

(22) Filed: Apr. 21, 2009

Related U.S. Application Data
(60) Provisional application No. 61/046,760, filed on Apr. 21, 2008.

(51) Int. Cl. A61K 39/00; C07K 14/005; C12N 15/00; C12N 5/06; C12N 15/87

(52) U.S. Cl. 424/185.1; 530/350; 435/320.1; 435/325; 435/455

(57) ABSTRACT

Novel recombinant vectors encoding viral antigens within a single vector or construct are disclosed. The vectors may be used to initiate immunological responses to antigens and to combat opportunistic and other infections. Viral antigens include those derived from CMV, EBV, adenovirus (Ad), Influenza A, herpes simplex, varicella, polyoma virus and respiratory viruses. A recombinant vector or construct may encode two antigenic peptides, three antigenic peptides (“TriVi”), or more than three antigenic peptides. Methods of making and other methods of using the novel recombinant vectors or constructs are also disclosed.

### PRIORITY CLAIM

The present application claims benefit to U.S. Provisional Patent Application No. 61/046,760, filed Apr. 21, 2008, the disclosure of which is incorporated herein by reference in its entirety.

### BACKGROUND

Cytomegalovirus (CMV) and Epstein Barr virus (EBV) are common herpes viruses that are acquired by a large number of the population by adulthood. Primary infection by either virus is typically asymptomatic and is self-limiting in healthy individuals. Infection persists in a latent state throughout the host's life, under control and surveillance of T lymphocyte mediated immunity against the viruses.

T lymphocytes are formed in the bone marrow, migrate to and mature in the thymus and then enter the peripheral blood and lymphatic circulation. T lymphocytes are subdivided into three distinct types of cells: helper T cells, suppressor T cells, and cytotoxic T cells. T lymphocytes, unlike B lymphocytes, do not produce antibody molecules, but express a heterodimeric cell surface receptor that recognizes peptide fragments of antigenic proteins that are attached to proteins of the major histocompatibility complex (MHC) and expressed on the surfaces of target cells; see, e.g., Abbas, A. K., Lichtman, A. H., and Pober, J. S., Cellular and Molecular Immunology, 1991.

Cytotoxic T lymphocytes (CTLs) are typically of the CD3+, CD8+, CD4− phenotype and lyse cells that display fragments of foreign antigens associated with class I MHC molecules on their cell surfaces. Target cells for CTL recognition include normal cells expressing antigens after infection by viruses or other pathogens; and tumor cells that have undergone transformation and are expressing mutated proteins or are over-expressing normal proteins.

Helper T cells are also CD3+ but can be distinguished from cytolytic T cells by expression of CD4 and absence of the CD8 membrane protein. CD4+ helper T cells recognize fragments of antigens presented in association with class II MHC molecules, and primarily function to produce cytokines that amplify antigen-specific T and B cell responses and activate accessory immune cells such as monocytes or macrophages. See, e.g., Abbas, A. K., et al., supra. Although most healthy individuals remain asymptomatic during their life, CMV and EBV can pose health risks in certain individuals and are associated with a number of clinical syndromes. CD4+ helper and CD8+ cytotoxic T lymphocytes are important components of the host immune response to viruses, bacterial pathogens and tumors. As a result, individuals with congenital, acquired or iatrogenic T cell immunodeficiency diseases may develop life threatening infections or malignancies. For example, both CMV and EBV are believed to possess oncogenic potential and are implicated in a number of human tumors, such as Burkitt's lymphoma, Hodgkin's disease, glioma cells and other brain tumors, sporadic epithelial carcinomas, certain unusual types of T cell lymphoma, Karposi's sarcoma, nasopharyngeal carcinoma and colorectal cancer.

Adoptive transfer of antigen-specific T cells to establish immunity has been demonstrated to be an effective therapy for viral infections and tumors in animal models (reviewed in Greenberg, P. D., Advances in Immunology (1992)). For adoptive immunotherapy to be effective, antigen-specific T cells usually need to be isolated and expanded in numbers by in vitro culture, and following adoptive transfer such cultured T cells must persist and function in vivo.

Although successful in application of adoptive immunotherapy, the production of viral-specific T cells is often problematic. Many culture systems have been developed to generate virus specific T cells, such as CMV- or EBV-infected or retrovirus-infected antigen presenting cells (APCs), including APCs derived from lymphoblastoid cell lines (LCLs) generated in vitro. (5, 6). These antigen presenting cells provide immunodominant virus-specific antigens. However there remains a risk of viral transmission when the cultured T cells are given to immunocompromised patients such as bone marrow transplant recipients. Thus, there remains a need for more effective and safe adoptive immunotherapy treatment of immunocompromised patients

Other strategies to produce virus-specific T cells safely include administration of virus-specific peptide antigens or purified viral protein-pulsed antigen presenting cells, but the high cost and only moderate efficacy of these methods are a concern. In addition, both CMV and EBV are often reactivated simultaneously, which may increase the severity of clinical symptoms beyond that by infection of one virus alone. (7).

Generation of multiple T cell lines that are specific for individual viruses is costly and laborious. Thus there remains a need for an immunotherapeutic treatment that is applicable for use against a multiplicity of viral infections within the same patient or within the same clinical presentation. The safe administration of such vaccines to patients not previously exposed to CMV, EBV or other similar virus is also provided.

### SUMMARY

Compositions, multi-specific lymphocytes and methods for their use in treating and preventing disease in a mammal are disclosed.

Composition comprising a vector encoding one or more viral antigens that are associated with opportunistic infections are also disclosed. For example, a recombinant DNA molecule encodes a fusion multiviral chimeric antigen comprising at least two viral antigens in order to create antigen-presenting cells (APC) and viral-specific T cells that are more efficient in fighting opportunistic infections in immunosuppressed individuals. Viral antigens include those derived from CMV, EBV, adenovirus (Ad), Influenza A, herpes simplex, varicella, polyoma virus, respiratory viruses as well as antigens commonly associated with opportunistic infections. Another aspect includes a recombinant DNA construct or vector that encodes a fusion multiviral chimeric antigen consisting of dual antigenic peptides. In another aspect of the invention, a recombinant DNA construct or vector encoding three antigenic peptides (“TriVi”) is provided. For example, a fusion multiviral chimeric antigen can comprise antigenic peptides from CMV pp65, EBV EBNA3C, and Influenza A MP1.

Methods of making peripheral blood mononuclear cells (PBMC) modified by plasmids encoding viral antigens are disclosed. Such modified PBMCs produce antigen presenting cells capable of eliciting CD4 and CD8 T cell responses to viral proteins.

A further aspect includes a pharmaceutical formulation comprising, consisting essentially of, or consisting of an in vitro expanded mammalian cytotoxic T lymphocyte (CTL) population. The CTL population may be genetically modified with at least one of the recombinant DNA constructs or vectors provided herein. The CTL population may be enriched prior to expansion for central memory T cells. The CTL population may be depleted prior to expansion of effector memory T cells.

A further aspect includes the use of a formulation as described herein for the preparation of a medicament for carrying out a method of treatment such as, for example, treatment of an infectious disease in a mammal, or treatment of cancer in a mammal.

In one aspect, a method of treating opportunistic viral infections by use of the autologous T cells modified to express more than one antigenic peptide, or having more than one antigenic specificity is provided. In another aspect, genetically engineered multi-specific antigen-presenting cells (APC) or antigen-specific T cells which express polypeptides having fusion multiviral chimeric antigens are provided. Such polypeptides include antigenic domains derived from one or more distinct class of virus.

T cells can be additionally modified to express a tumor-specific chimeric immunoreceptor in order to increase the efficacy of the adoptive immunotherapy.

### BRIEF DESCRIPTION OF THE DRAWINGS

FIG. 1 shows pp65 immunochemical staining for EBNA3Ĉ-pp65 gene in 293T cells-293T cells were plated on 6 well plate and transfected with EBNA3Ĉ-pp65 gene. Immunochemical staining was performed according to the ABC technique—pp65 Ab→biotinylated secondary Ab→preformed Avid in and Biotinlyated horseradish peroxidase macromoleular Complex→H₂O₂/AEC→bred nucleus. The cells were fixed and washed, then stained with primary CMVpp65 antibody, followed by secondary biotinylated antibody and then a preformed avidin and biotinylated horseradish peroxidase macromolecular complex. After incubating with hydrogen peroxide, pp65 was visualized by staining with AEC. The positive nuclear staining of the EBNA3Ĉ-pp65 cells indicates expression of pp65 antigen.

FIG. 2 shows a plasmid map of the TriVi plasmid construct of antigenic peptides from CMV pp65, EBV EBNA3C, and Influenza A MP1 virus (i.e., the EBNA3Ĉ-pp65̂k-MP1 gene), with the adenovirus expression vector—pLP-Adeno-X-CMV.

FIG. 3. pp65 immunochemical staining for EBNA3CA-pp65̂k-MP1 gene in 293T cells. 293T cells were transfected with EBNA3Ĉ-pp65 gene. Immunochemical staining was performed according to the ABC technique described above. Positive nuclear staining of the EBNA3Ĉ-pp65̂k-MP1 cells indicate expression of pp65 viral antigen by the transfected 293T cells.

FIG. 4 shows a Western blot for EBNA3Ĉ-pp65 and EBNA3Ĉ-pp65̂k-MP1 gene in 293 T cells—gene transfected 293T cells (pJ01741 (EBNA3Ĉ-pp65_pEK) and pJ01942 (EBNA3Ĉ-pp65̂k-MP1_pDNR-CMV)) were lysed and the proteins loaded on the acrylamide gel. After transfer the protein to the nitrocellulose membrane, pp65 and MP1 antibody was probed individually. Lane 1—protein ladder. Lane 2—pJ01950#1 (pDNR-CMV). Lane 3—pJ01942#8 (EBNA3Ĉ-pp65̂k-MP1_pDNR-CMV), 119 kD. Lane 4—pJ01741#1 (EBNA3Ĉ-pp65_pEK), 92 kD. The fusion protein size for EBNA3Ĉ-pp65 is 92 kD, and for EBNA3Ĉ-pp65̂k-MP1 is 119 kD. Bands of the expected fusion protein sizes are seen in both lanes 3 and 4, indicating successful expression of the CMV pp65 (left gel) and influenza virus MP1 (right gel) antigens.

FIG. 5. EBNA3Ĉ-pp65̂k-MP1_pLP-Adeno-X-CM adenovirus was transfected into 293T cells and run on a Western blot with pp65 antibody. 5 clones with different multiplicity of infection (MOI) (1, 5, 10) were used. Gel 1: Lane 1-3 clone # A MOI 1, 5, 10; Lane 4-6 clone # B MOI 1, 5, 10; Lane 7-9 clone # C MOI 1, 5, 10; Lanel0 pJ01942 positive control; Lane 11 negative control MOI 1. Gel 2: Lanel-3 clone # D MOI 1, 5, 10; Lane 4, 8, 6 clone # E MOI 1, 5, 10; Lane 7, 5, 9 negative control MOI 1, 5, 10; Lane 10 pJ01942 positive control. A band of the expected fusion protein size (119 kDa) indicates successful expression of the CMV pp65 antigen in all clones.

FIG. 6 shows an exemplary Western blot probed with MP1 antibody of 293T cells transfected with EBNA3Ĉ-pp65̂k-MP1_pLP-Adeno-X-CMV adenovirus. Five clones having different MOI (1, 5, 10) were used. All clones expressed MP1 gene. Gel 3: Lanes 1-3 clone # A MOI 1, 5, 10; Lanes 4-6 clone # B MOI 1, 5, 10; Lanes 7-9 clone # C MOI 1, 5, 10; Lane 10 pJOl 942 positive control; Lane 11 negative control MOI 1. Gel 4: Lane 1-3 clone # D MOI 1, 5, 10; Lane 4, 8, 6 clone # E MOI 1, 5, 10; Lanes 7, 5, 9 control MOI 1, 5, 10; Lanel0 pJ01942 positive control. A band of the expected fusion protein size (119 kDa) indicates successful expression of the influenza virus MP1 antigen in all clones.

FIG. 7 shows a plasmid map of EBNA3Ĉ-pp65̂k gene with an adenovirus expression vector—AD5/35 FL 9674-2A-9666 WT FoKI.

FIG. 8 shows a Western blot of K₅₆₂ cells transfected with EBNA3Ĉ-pp65̂k_AD5/35 FL 9674-2A-9666 WT FoKI adenovirus and probed with pp65 antibody. Host cells having different MOI (30, 100, 300, 1000) were used. All clones expressed pp65 gene. AD5/35 EBNA3 Cpp65: 1. K562 control; 2. K562 MOI 30; 3. K562 MOI 100; 4. K562 MOI 300; 5. K562 MOI 1000; 6. 293T pJ01741#1(EBNA3Ĉ-pp65̂k-pEK(7-11-07). EBNA3 Cpp65—92 kb. β-actin—42 kb.

FIG. 9 shows successful generation of viral specific CD8+ T cells using v-APC generated with EBNA3C:pp65:MP-1_Ad5/F35. Demonstration that TriVi-Ad5 transfected autologous monocytes elicit CD8 T cell responses to CMVpp65, EBV EBNA3C, Influenza A MP-1 in single cultures by tetramers. T cells that are specific for different viral antigens were analyzed by flow cytometry using anti-CD8 antibody and the indicated tetramers on day 14 of the REM stimulation: the Multi-Allele Negative tetramer is shown in the upper right hand graph; CMVpp65/HLA-A2 tetramer (NLVPMVATV; SEQ ID NO: 1) is shown in the middle left hand graph; EBNA3C/HLA A2 tetramer (LLDFVRFMGV; SEQ ID NO: 2) is shown in the middle right hand graph; and MP1/HLA-A2 tetramer (GILGFVFTL; SEQ ID NO: 3) is shown in the lower left hand graph.

FIG. 10 shows TriVi-Ad5 transfected autologous monocytes elicit CD8 T cell responses to CMVpp65, EBV EBNA3C, Influenza A MP-1 in single cultures by tetramers as exemplified by flow cytometry. The upper right corner of each scatter plot represents CD8+ tetramer+ cells.

FIG. 11 shows a CMVpp65 expression plasmid map. The DNA vector was used to genetically modify donor PBMC to express CMVpp65 antigen.

FIG. 12 shows an exemplary experimental design for ex vivo expansion of CMV specific T cells.

FIG. 13 shows flow cytometry analysis of gene modified PBMC (T-APC). Donor PBMC were electroporated in the presence of CMVpp65 plasmid or GFP plasmid (control). Control T-APC were analyzed by flow cytometry for GFP expression.

FIG. 14 shows flow cytometry analysis of gene modified PBMC following stimulation. Rapid expansion of CMV specific T cells using gene modified PBMC as T-APCs. Purified central memory T cells (CD45RO+CD62L+Tcm) were co-cultured with irradiated T-APC and re-stimulated weekly. High levels of CMVpp65/HLA-A2 tetramer+CD8+ cells were detected by flow cytometry after 3rd stimulation.

FIG. 15 shows CMV pp65-specific cytotoxicity. 4 hr chromium release assay was performed using CMV specific T cells derived from FACS sorted TCM cells as Effectors, and autologous LCL (lymphoblastoid cell line) loaded with either pp65 or irrelevant peptide as targets.

FIG. 16 shows a map of an EBNA3C:pp65 expression plasmid. The DNA vector was used to genetically modify donor PBMC to co-express EBV EBNA3C and CMVpp65 antigens.

FIG. 17 shows an exemplary experimental design for a simultaneous ex vivo expansion of EBV and CMV specific T cells.

FIG. 18 shows an exemplary growth curve of ex vivo expanded EBV/CMV specific T cells. Viral antigen presenting cells (v-APC, a.k.a. T-APC) were generated by transfecting donor PBMC with EBNA3C:pp65pEK plasmid (pj 01741) using standard electroporation procedures. Unfractionated PBMC were co-cultured with irradiated EBNA3C:pp65 expressing T-APC and re-stimulated weekly. Cell numbers after first (S1) and second (S2) stimulations are depicted. Cultures stimulated with EBNA3C expressing T-APC were used as a control (top line). After the second stimulation, an increase in both CD4 (S2D4) and CD8 (S2D8) cells were observed.

FIG. 19 shows an exemplary flow cytometry analysis of interferon-gamma (IFN-γ) production upon EBV/CMV antigen stimulation. EBV/CMV specific T cells after chimeric EBNA3C:pp65 T-APC stimulation (exp. 1) and subsequent enrichment by IFN-γ capture (exp. 2), were co-cultured with stimulators for 16 hours. Intracellular IFN-γ+ cells responsive to medium (control), autoLCL (EBV presenting) and pp65 peptide mix loaded autoLCL (EBV+CMV presenting) are depicted.

FIG. 20 shows EBV-specific cytotoxicity. A four hour chromium release assay was performed using EBV/CMV specific T cells derived from 2nd T-APC stimulation as effectors and autologous LCL (top line) as targets. LCL from HLA mismatched donor (lower line) were used as negative targets.

FIG. 21 shows flow cytometry analysis of CMV specific T cells generated with chimeric EBNA3C:pp65 T-APC stimulation. CMVpp65/HLA-A2 tetramer+ CD8+ cells were detected using flow cytometry after 2nd stimulation with irradiated T-APC.

FIG. 22 shows flow cytometry analysis of EBV/CMV specific T cells derived from PBMC contain high numbers of TCM cells. EBV/CMV T cells after 2nd stimulation with T-APC were analyzed with FACS for central memory markers. More than 70% of the cells express CD62L.

FIG. 23 depicts the nucleotide and amino acid sequence for EBNA3C-pp65. The sequence order is EBNA3C(1-129)-pp65̂k-EBNA3C(159-300). SEQ ID NO: 4 is the coding strand of DNA. SEQ ID NO: 5 is the complementary strand of DNA. SEQ ID NO: 6 is the amino acid sequence. EBNA3Ĉ: EBNA3C with deletion of binding and regulation domain for retinoblastoma (deleted amino acids 130 to 158, 29 amino acids) (underlined). pp65̂k: pp65 with deletion of kinase activity (deleted 436-438, 3 amino acids).

FIG. 24 depicts the nucleotide and amino acid sequence for EBNA3C-pp65. The sequence order for EBNA3C-pp65-MP1 is EBNA3C(1-129)-pp65̂k-EBNA3C(159-300)-MP1. SEQ ID NO: 7 is the coding strand of DNA. SEQ ID NO: 8 is the complementary strand of DNA. SEQ ID NO: 9 is the amino acid sequence of EBNA3C-pp65̂k-MP1. EBNA3Ĉ: EBNA3C with deletion of binding and regulation domain for retinoblastoma (deleted amino acids 130 to 158, 29 amino acids) (underlined text). pp65̂k: pp65 with deletion of kinase activity (deleted 436-438, 3 amino acids) (light text). Dark text=MP1.

FIG. 25 depicts the nucleotide and amino acid sequence for EBNA3C-pp65. The sequence order for EBNA3C-pp65-MP1 is EBNA3C(1-129)-pp65̂k-EBNA3C(159-300)-MP1. SEQ ID NO: 7 is the coding strand of DNA. SEQ ID NO: 8 is the complementary strand of DNA. SEQ ID NO: 9 is the amino acid sequence. EBNA3Ĉ-pp65̂k-MP1. EBNA3Ĉ: delete EBNA3C binding and regulation domain for retinoblastoma (delete amino acid 130 to 158, 29 amino acids) (underlined text). pp65̂k: delete kinase activity (delete 436-438, 3 amino acids) (light text). Dark text=MP1.

FIG. 26 shows an exemplary 4-hour chromium release assay. Viral specific CD8+ T cells were successfully generated using v-APC generated with EBNA3C:pp65-pEK. A 4-hour chromium release assay was performed using EBNA3C/pp65-specific T cells derived from 2nd v-APC stimulation as effectors and autologous EBV-transformed LCL (top line), expressing the EBV antigen EBNA3C as targets. LCL from HLA mismatched allogenic donor (bottom line) were used as negative targets. Thus, the EBNA3C/pp65-specific T cells specifically kill EBV expressing targets in the context of autologous HLA/MHC class I.

FIG. 27 shows generation of viral specific CD8+ T cells using v-APC generated with EBNA3C:pp65_Ad5/F35. Viral antigen presenting cells (v-APC) were generated by transducing donor derived monocytes with codon optimized Ad5/35 EBNA3C:pp65 [EBNA3C:pp65(CO)](pj02258). Unfractionated PBMC were co-cultured with the irradiated v-APC and re-stimulated weekly. The resultant cells from the first antigen stimulation were expanded with conventional rapid expansion medium (REM) containing irradiated PBMC, irradiated LCL and OKT3. 14 days after REM, T cells were left unstimulated or stimulated with a pp65-peptide mix, and viral-specific IFN-γ production by CD8+ T cells was determined by flow cytometry. IFN-γ expressing CD8+ T cells (7.67%) were observed after stimulation with the pp65-peptide mix.

FIG. 28 shows generation of viral specific CD8+ T cells using v-APC generated with EBNA3C:pp65_pEK. Viral antigen presenting cells (v-APC) were generated by transfecting donor derived monocytes with codon optimized EBNA3C:pp65_pEK plasmid [EBNA3C:pp65(CO)](pj02290). Unfractionated PBMC were co-cultured with irradiated v-APC that express EBNA3C:pp65 and re-stimulated weekly. The resultant cells from the first antigen stimulation were expanded with conventional rapid expansion medium (REM) containing irradiated PBMC, irradiated LCL and OKT3. 14 days after REM, T cells were left unstimulated or stimulated with a pp665-peptide mix, and viral-specific IFN-γ production by CD8+ T cells was determined by flow cytometry. Here, too, IFN-γ expressing CD8+ T cells (6.02%) were observed after stimulation with the pp65-peptide mix.

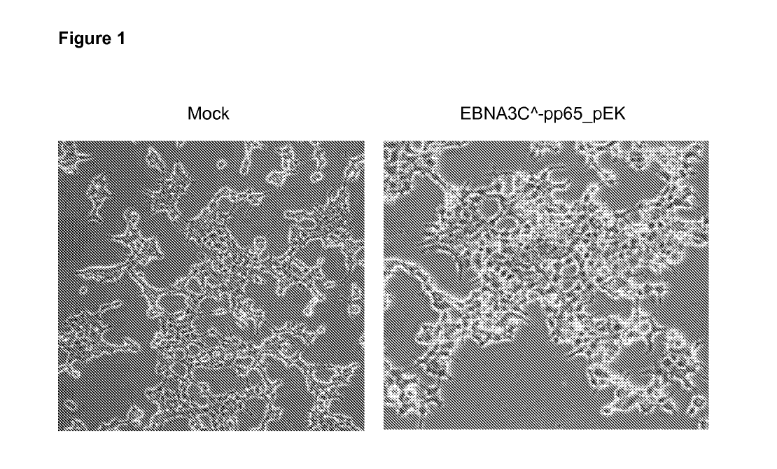

### DETAILED DESCRIPTION

Cytomegalovirus (CMV) and Epstein-Barr virus (EBV) are generally associated with opportunistic infections and malignancies in immunosuppressed individuals, such as HIV/AIDS patients, recipients of allogenic stem cell transplantation, bone marrow transplantation, or other organ transplant. Immunosuppressed individuals have little to no viable T cells that guard against primary or reactivated infection. Thus, these individuals are more susceptible to life-threatening infection. Within this population, seronegative patients undergoing transplantation are particularly susceptible to more serious complications that subsequently result from a primary CMV and EBV infection due to their lack of prior immunity against these opportunistic viruses.

“T cells” or “T lymphocytes” as used herein may be from any mammal, preferably a primate species, including monkeys, dogs, cats, and humans. In some embodiments the T cells are allogenic (from the same species but different donor) as the recipient subject; in some embodiments the T cells are autologous (the donor and the recipient are the same); in some embodiments the T cells are syngeneic (the donor and the recipients are different but are genetically identical).

Cytotoxic T lymphocyte (CTL) as used herein refers to a T lymphocyte that expresses surface CD8 (i.e., a CD8+ T cell). In some embodiments such cells are “memory” T cells (T_{M} cells) that are antigen-experienced. “Central memory” T cell (or “T_{CM}”) as used herein refers to a CTL that expresses CD62L on the surface thereof (i.e., CD62L+CD8+ cells).

“Effector memory” T cell (or “T_{EM}”) as used herein refers to a CTL that does not express CD62L on the surface thereof (i.e., CD62L− CD8+ cells).

“Enriched” and “depleted” as used herein refers to amounts of cell types in a mixture or to the processing of a mixture of lymphocytes to a process or step which results in an increase in the number of the “enriched” type and a decrease in the number of the “depleted” cells. Thus, depending upon the source of the original population of cells subjected to the enriching process, a mixture or composition may contain 60, 70, 80, 90, 85, or 99 percent or more (in number or count) of the “enriched” cells and 40, 30, 20, 10, 5 or 1 percent or less (in number or count) of the “depleted” cells.

The induction, augmentation, and manipulation of antiviral immunity have many applications clinical applications. The ex vivo genetic modification of lymphoid cells to express viral antigen transgenes stimulates T-cell responses in vitro and allows for selection of antiviral T cells. These selected T cells having specific viral recognition are then useful for infusion into a host patient. This method is commonly referred to as adoptive immunotherapy of infectious diseases. In addition, genetic modification of T cells to express chimeric antigen receptors has application in cancer therapy. Viral transgene expressing lymphoid cells when re-administered to patients after receiving adoptive therapy with virus specific T cells will serve to activate and expand these T cells in vivo.

As used herein, an antigen presenting cell (APC) include T cells, dendritic cells, B cells, macrophages and other lymphocytes.

As used herein, peripheral blood mononuclear cells (PBMC) include autologous and allogeneic cells PBMC.

As used herein, an activated T cell is a T cell that has received at least two mitogenic signals. Activated T cells can be identified phenotypically, for example, by virtue of their expression of CD25. Cells that express the IL-2 receptor (CD25) are referred to herein as “activated”. A pure or highly pure population of activated cells typically express greater than 85% positive for CD25. Other markers/phenotypes that are indicative of T cell activation include production of IL-2 and IFN-γ, as well as cytotoxic effector activity.

As used herein, T cells for adoptive immunotherapy refer to any T cells that have been treated for use in adoptive immunotherapy. Examples of such cells include any T cells prepared for adoptive immunotherapy. These T cells are often genetically manipulated to increase their therapeutic efficacy, and expanded ex vivo to numbers that are significant enough for transfer into an adult human.

One embodiment is directed to a novel recombinant DNA molecule that encodes a fusion multiviral chimeric antigen consisting of three antigenic peptides (“TriVi”): CMV pp65, EBV EBNA3C, and Influenza A MP1. The recombinant DNA encoding the TriVi plasmid construct was designed de novo for the simultaneous expression of a single polypeptide that contains domains of CMV pp65, EBV EBNA3C, and Influenza A MP-1. In another embodiment, the TriVi or other anti-viral plasmid construct may be used to generate mammalian viral-specific T cells. In some embodiments, T cells having dual reactivity to viral antigen are provided. For example, a dual viral antigen construct may be used to generate T cells specific to CMV, EBV, or Influenza, or any combination thereof. Also, T cells derived from donors who were CMV and EBV immune, generated activated T cells having dual reactivity to pp65 and EBNA3C.

Adoptive transfer of anti-viral T cells can reconstitute immunity and protect from viral disease. Peripheral blood mononuclear cells (PBMC) genetically modified by electrotransfer with plasmid vectors that encode viral antigen transgenes can be used to produce autologous antigen presenting cells (APCs) capable of eliciting CD4 and CD8 T cell responses to viral proteins, such as Cytomegalovirus pp65 (CMV pp65) and Epstein Barr virus EBNA3C (EBV EBNA3C). Other methods can also be employed for transducing T lymphocytes. Such methods include, for example, use of retroviral vectors (e.g., lentiviral vectors) or adnoviral vectors for transduction.

PBMC modified to express viral antigen transgenes using plasmid electrotransfer can act as powerful T-APC to induce robust viral specific T cell expansion. Accordingly, one embodiment is to efficiently expand T lymphocytes to large numbers in vitro. Such rapidly expanded T cell populations can be used, inter alia, for infusion into individuals for the purpose of conferring a specific immune response, as exemplified herein. The T cells can be either CD8+ cytotoxic T cells or CD4+ helper T cells, and they can react with antigens encoded in any of a variety of virally infected cells or tumor cells.

PBMC and T-Cell Culture. One aspect of certain embodiments provides methods for rapidly expanding populations of T lymphocytes, including human cytotoxic T lymphocytes and helper T lymphocytes, which can be particularly useful in adoptive immunotherapy of human diseases.

T cells can be obtained from any suitable source, including, but not limited to spleen tissue, lymph nodes, peripheral blood, tumors, ascitic fluid, dermal biopsies, and CNS fluids. Any method for harvesting T cells from the host can be used. For example, Ficoll-Paque (commercially available from Pharmacia) centrifuged peripheral blood mononuclear cells (PBMC) can be used. Alternatively, purified CD4+ or CD8+ T cells isolated by immunoaffinity procedures, such as through the use of MACs or dyna-beads, can be used. Appropriate methods for obtaining T cells are taught, for example, in Tuting, et al., J. Immunol. 160: 1139-1147 (1998).

The T cells are referred to as “target T cells”. In general, target T cells are added in small numbers to a culture vessel and standard growth medium that has been supplemented with: (i) an appropriate amount of antibody directed at the CD3 component of the T cell receptor complex to provide the T cell receptor signal; and (ii) a disproportionately large number of feeder cells, preferably γ-irradiated PBMC as described below, which provide co-stimulatory signals. Preferably, human recombinant IL-2 or another suitable IL-2 preparation is added in low concentrations at 3-5 day intervals (typically on day 1, on day 5 or 6, and on day 8 or 9). The method results in a rapid expansion of T cells, typically in the range of a 500- to 3000-fold expansion in 8 to 14 days. The present method is thus approximately 100- to 1000-fold more efficient for each stimulation cycle than currently described methods of culturing human T cells

Methods for efficient expansion T cell clones for use in adoptive immunotherapy, are known in the art and include, for example, those described by Riddell et al. (Science, 257:238-240, 1992) which is incorporated herein by reference. This method dramatically shortens the time required to grow the numbers of cells required to modulate human immunity.

The source of cells to be transduced with the vectors and plasmids provided (i.e., the target T cells) can be obtained from the subject to be treated. Alternatively, T cells can be obtained from persons other than the subject to be treated provided that the recipient and transferred cells are immunologically compatible. The source cells can be obtained from immunosuppressed individuals. Typically, the cells are derived from tissue, bone marrow, fetal tissue, or peripheral blood. Preferably, the cells are derived from peripheral blood. If the T cells are derived from tissues, single cell suspensions should be prepared using a suitable medium or diluent. The generation of polyclonal populations of T cells is described

Fusion multiviral chimeric antigen. DNA and protein constructs that are useful as vaccines can be used prophylactically or in persons already exposed to or infected with one or more opportunistic and oncogenic virus. Uses for these constructs include methods for augmenting immune responses to these viruses, vaccinating against the viruses, diagnosing the viruses, producing activated T cells that recognize the viruses and producing antigen presenting cells that present the virus epitopes using methods described above.

One embodiment is directed to a recombinant DNA construct encoding one or more viral antigens. The selected viral antigens are those associated with opportunistic infections arising after stem cell, bone marrow or organ transplants. Viral antigens associated with anti-tumor activity are also included herein. Preferably, the recombinant DNA molecule encodes a fusion multiviral chimeric antigen comprising at least two antigens (dual antigen). One example of such a vector is provided as SEQ ID NO: 10 (EBNA3C-pp65 vector sequence). Also provided are recombinant DNA vectors encoding at least three viral antigens in order to create APCs and viral-specific T cells that are more efficient in fighting opportunistic infections in immunosuppressed individuals. For example, a TriVi construct providing the simultaneous expression of a single polypeptide containing domains of CMV pp5, EBV EBNA3C, and Influenza A MP-1 is provided as SEQ ID NO:7. The chimeric polypeptide sequence is provided as SEQ ID NO: 9. In one embodiment, a multiviral chimeric antigen construct may include two viral antigens containing domains of CMV, EBM or Influenza or any combination thereof. An exemplary construct is provided as SEQ ID NO:4. The encoded amino acid sequence of the dual viral chimeric antigen is provided as SEQ ID NO: 6. Multi-viral antigens allow for the culture of a single specific T-cell population thereby reducing the cost of developing treatment for opportunistic infection. A preselected or predesired population of activated T-cells having specificity to opportunistic and infectious viruses is also obtained thereby increasing the efficiency and efficacy of developing and preparing such compositions for use in immunotherapic medicaments.

Viral antigens that may be encoded are not limited to pp65, EBNA3C or MP-1. Suitable viruses from which antigens may be encoded include those associated with common opportunistic infections. For example, CMV, EBV, adenovirus (Ad), Influenza A, herpes simplex, varicella, polyoma virus, and other respiratory or infectious viruses.

CMV encodes many immunogenic peptides. T cells are known to target the immediate-early protein, virion envelope glycoprotein B, and the internal matrix proteins pp65 and pp150. CMV pp65 is the immunodominant antigen, targeted by 70% to 90% of CMV-specific T cells (6). The immune response to EBV appears to follow a hierarchy of immunodominance among eight latent proteins: EBNA1, EBNA2, EBNA3A, EBNA3B, EBNA3C, EBNALP and LMPs 1 and 2. The EBNA3 family of nuclear proteins represents the dominant target antigen (8). EBV EBNA3C is nuclear antigen 3C of the Epstein-Barr virus, which is a regulatory transcription factor.

Influenza A MP-1 is a matrix protein of the influenza virus. The well-characterized protein MP1 from influenza A is a convenient target antigen since it is also an opportunistic virus, and from a young age almost all individuals have immunity to influenza and therefore have responsive circulating memory T cells. Furthermore, because the cellular immune responses to MP1 in HLA-A2 individuals usually responds to an immunodominant epitope (amino acid 58-66), tetramer technology can readily identify MP1-specific T cells making isolation and identification easier, for example using fluorescence activated cell sorting.

The fusion multiviral chimeric antigen may incorporate any antigenic peptide from any opportunistic or non-opportunistic virus. In some aspects the fusion multiviral chimeric antigen may comprise three or more antigenic peptides from the same virus. Alternatively, the fusion multiviral chimeric antigen may comprise three or more antigenic peptides, each from different viruses. Any combination of viral antigenic peptides may be used.

A preferred embodiment is directed to a novel recombinant DNA molecule or construct referred to herein as “TriVi”. TriVi encodes a fusion multiviral chimeric antigen consisting of three antigenic peptides: CMV pp65, EBV EBNA3C, and Influenza A MP-1 [SEQ ID NO: 9]. The recombinant DNA encoding the TriVi plasmid construct was designed de novo for the simultaneous expression of a single polypeptide that contains domains of CMV pp65, EBV EBNA3C, and Influenza A MP-1. CMV pp65 is a lower matrix protein in human CMV. The codon optimized cDNA consists of a kinase modified pp65 flanked by AA amino acids 1-129 of EBNA3C on its N-terminal and AA's 159-300 on its C-terminal, thus eliminating the AA sequence 130-158 implicated in perturbation of cell cycle control. To this chimera Influenza A MP-1 was fused to the C-terminal resulting in the full length TriVi construct.

The TriVi or other anti-viral plasmid construct may be used to generate mammalian viral-specific T cells and APCs using methods described herein. Preferably, the viral-specific T cells have specificity to all three viruses contained in the TriVi plasmid construct. TriVi-specific T cells are generated from PBMC feeder cells genetically modified to produce APCs and target T cells as described in Example 4 below. The PBMC feeder cells are preferably transfected by standard electroporation procedures. The non-viral electrotransfer of a recombinant protein derived from a viral pathogen avoids potential infection that can be associated with use of whole virus.

The TriVi specific T cells can be additionally modified to express a tumor-specific chimeric immunoreceptor in order to increase the efficacy of the adoptive immunotherapy. Examples of these bispecific anti-viral and anti-tumor T-cells and be found in U.S. patent application Ser. No. 11/700,762, filed Feb. 1, 2007, which is incorporated herein in its entirety by reference.

Genetic modification of PBMC. In further embodiments, the PBMC are genetically modified in order to introduce additional functional genes to be used in immunotherapy. There are a number of different circumstances in which the introduction of functional genes into T cells to be used in immunotherapy are desirable. For example, the introduced gene or genes may improve the efficacy of therapy by promoting the viability and/or function of transferred T cells; or they may provide a genetic marker to permit selection and/or evaluation of in vivo survival or migration; or they may incorporate functions that improve the safety of immunotherapy, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al., describing the use of bifunctional selectable fusion genes derived from fusing a dominant positive selectable marker with a negative selectable marker. Various infection techniques have been developed which utilize recombinant infectious virus particles for gene delivery. The viral vectors which have been used in this way include virus vectors derived from simian virus 40 (SV40; Karlsson et al., Proc. Natl. Acad. Sci. USA 84 82:158, 1985), adenoviruses (Karlsson et al., EMBO J. 5:2377, 1986), adeno-associated virus (AAV) (B. J. Carter, Current Opinion in Biotechnology 1992, 3:533-539), and retroviruses (Coffin, 1985, pp. 17-71 in Weiss et al. (eds.), RNA Tumor Viruses, 2nd ed., Vol. 2, Cold Spring Harbor Laboratory, New York). Thus, gene transfer and expression methods are numerous but essentially function to introduce and express genetic material in mammalian cells. Several of the above techniques have been used to transduce hematopoietic or lymphoid cells, including calcium phosphate transfection (Berman et al., supra, 1984), protoplast fusion (Deans et al., supra, 1984), electroporation (Cann et al., Oncogene 3:123, 1988), and infection with recombinant adenovirus (Karlsson et al., supra; Reuther et al., Mol. Cell. Biol. 6:123, 1986), adeno-associated virus (LaFace et al., supra) and retrovirus vectors (Overell et al., Oncogene 4:1425, 1989). Primary T lymphocytes have been successfully transduced by electroporation (Cann et al., supra, 1988) and by retroviral infection (Nishihara et al., Cancer Res. 48:4730, 1988; Kasid et al., supra, 1990; and Riddell, S. et al., Human Gene Therapy 3:319-338, 1992).

Retroviral vectors provide a highly efficient method for gene transfer into eukaryotic cells. Moreover, retroviral integration takes place in a controlled fashion and results in the stable integration of one or a few copies of the new genetic information per cell.

Retroviruses are a class of viruses which replicate using a virus-encoded, RNA-directed DNA polymerase, or reverse transcriptase, to replicate a viral RNA genome to provide a double-stranded DNA intermediate which is incorporated into chromosomal DNA of an avian or mammalian host cell. Most retroviral vectors are derived from murine retroviruses. Retroviruses adaptable for use can, however, be derived from any avian or mammalian cell source. These retroviruses are preferably amphotropic, meaning that they are capable of infecting host cells of several species, including humans. A characteristic feature of retroviral genomes (and retroviral vectors used as described herein) is the retroviral long terminal repeat, or LTR, which is an untranslated region of about 600 base pairs found in slightly variant forms at the 5′ and 3′ ends of the retroviral genome. When incorporated into DNA as a provirus, the retroviral LTR includes a short direct repeat sequence at each end and signals for initiation of transcription by RNA polymerase II and 3′ cleavage and polyadenylation of RNA transcripts. The LTR contains all other cis-acting sequences necessary for viral replication.

A “provirus” refers to the DNA reverse transcript of a retrovirus that is stably integrated into chromosomal DNA in a suitable host cell, or a cloned copy thereof, or a cloned copy of unintegrated intermediate forms of retroviral DNA. Forward transcription of the provirus and assembly into infectious virus occurs in the presence of an appropriate helper virus or in a cell line containing appropriate sequences enabling encapsidation without coincident production of a contaminating helper virus. Mann et al. (Cell 33:153, 1983) describe the development of cell lines (e.g., .PSI.2) which can be used to produce helper-free stocks of recombinant retrovirus. These cells lines contain integrated retroviral genomes, which lack sequences required in cis for encapsidation, but which provide all necessary gene product in trans to produce intact virions. The RNA transcribed from the integrated mutant provirus cannot itself be packaged, but these cells can encapsidate RNA transcribed from a recombinant retrovirus introduced into the same cell. The resulting virus particles are infectious, but replication-defective, rendering them useful vectors which are unable to produce infectious virus following introduction into a cell lacking the complementary genetic information enabling encapsidation. Encapsidation in a cell line harboring transacting elements encoding an ecotropic viral envelope (e.g., .PSI.2) provides ecotropic (limited host range) progeny virus. Alternatively, assembly in a cell line containing amphotropic packaging genes (e.g., PA317, ATCC CRL 9078; Miller and Buttimore, Mol. Cell. Biol. 6:2895, 1986) provides amphitropic (broad host range) progeny virus. Such packing cell lines provide the necessary retroviral gag, pol and env proteins in trans. This strategy results in the production of retroviral particles which are highly infectious for mammalian cells, while being incapable of further replication after they have integrated into the genome of the target cell. The product of the env gene is responsible for the binding of the retrovirus to viral receptors on the surface of the target cell and therefore determines the host range of the retrovirus. The PA 317 cells produce retroviral particles with an amphotropic envelope protein, which can transduce cells of human and other species origin. Other packaging cell lines produce particles with ecotropic envelope proteins, which are able to transduce only mouse and rat cells.

Many gene products have been expressed in retroviral vectors. This can either be achieved by placing the sequences to be expressed under the transcriptional control of the promoter incorporated in the retroviral LTR, or by placing them under the control of a heterologous promoter inserted between the LTRs. The latter strategy provides a way of coexpressing a dominant selectable marker gene in the vector, thus allowing selection of cells which are expressing specific vector sequences.

It is contemplated that overexpression of a stimulatory factor (for example, a lymphokine or a cytokine) may be toxic to the treated individual. Therefore, included are gene segments that cause the T cells to be susceptible to negative selection in vivo. By “negative selection” is meant that the infused cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene (Wigler et al., Cell 11:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

As described in Riddel et al., the T lymphocyte pool from which T cells for adoptive immunotherapy could potentially be isolated contains CD45RA+ CD62L+ naïve (Tn), CD45RO+ CD62L+central memory (Tcm), and CD62L− effector memory (Tem) subsets that differ in phenotype, function, and homing. After recognition of antigen in vivo, Tn cells undergo proliferation and differentiation, resulting in the generation of large numbers of CD62L− effector T cells (Te), most of which die as antigen is cleared leaving a small pool of Tcm and Tem cells. Memory T cells respond to antigen re-exposure in vivo and in vitro by differentiating again into Te cells. The lifelong maintenance of T cell memory suggests that some cells in the memory pool may be capable of both self-renewal and differentiation, and there is evidence in mice that a subset of memory T cells may be endowed with stem cell like properties Mononuclear cells containing the T lymphocytes are isolated from the heterogenous population according to any of the methods well known in the art. As illustrative examples, Ficoll-Hypaque gradient centrifugation, fluorescence-activated cell sorting (FACs), panning on monoclonal antibody coated plates, and/or magnetic separation techniques can be used (separately or in combination) to obtain purified populations of cells for expansion. Antigen-specific T cell clones are isolated by standard culture techniques known in the art involving initial activation of antigen-specific T cell precursors by stimulation with antigen-presenting cells and subsequent cloning by limiting dilution cultures using techniques known in the art, such as those described in Riddell and Greenberg (J. Immunol. Meth., 128:189-201, 1990); and Riddell et al. (J. Immunol., 146:2795-2804, 1991). See also, the Examples below. The T cell clones isolated in microwells in limiting dilution cultures typically have expanded from a single cell to 2×10̂4 to 5×10̂5 cells after 14 days. At this time individual clones are placed in appropriate culture media in plastic culture vessels with disproportionately large numbers of feeder cells which provide co-stimulatory functions, and, preferably, anti-CD3 monoclonal antibody to provide T cell receptor stimulation. This initial phase of rapid expansion when the clone is transferred from a microwell is generally carried out in a culture vessel, the size of which depends upon the number of target cells, and which may typically be a 25 cm̂2 flask. The size of the culture vessel used for subsequent cycles of T cell expansion depends on the starting number of T cells and the number of cells needed (usually for therapeutic use). Typical starting cell numbers for different sized culture vessels are as follows: 5×10̂4 to 2×10̂5—approximately 25 cm̂2 flask; 2×10̂5 to 5×10̂5-approximately 75 cm̂2 flask; 5×10̂5 to 1×10̂6—approximately 225-cm̂2 flask; and 1×10̂6 to 2×10̂6—roller bottle. The approximate initial volume of media used with each flask is: 25 cm̂2—20-30 ml; 75 cm̂2—60-90 ml; 225 cm̂2—100-200 ml; roller bottle—500 ml.

As illustrated below, studies using 10 different T cell clones of varying antigen specificities, that were initially derived from 4 different human donors, indicate that a 500- to 3000-fold expansion in clonal T cell number (mean, 1200-fold) can be readily achieved within a single 10-13 day cycle of growth using a rapid expansion method (see Example 1).

As illustrated in Example 2, the rapid expansion method can be readily scaled up to produce large numbers of antigen-specific T cells (greater than 10̂9 cells) for use in adoptive immunotherapy. In that example, large numbers of CMV-specific and HIV-specific T cells were generated from 10 clones initially derived from 6 different human donors.

As used herein, “feeder cells” are accessory cells (such as the preferred γ-irradiated PBMC and LCL cells) that provide co-stimulating functions in conjunction with T cell receptor activation (which can be achieved by ligation of the T cell receptor complex with anti-CD3 monoclonal antibody). The feeder cells may be from a seropositive or seronegative individual. In one aspect, a seronegative donor is preferred in order to reduce the risk of infection of immunosuppressed individuals who have not been exposed to the targeted viruses using the TriVi plasmid construct or other viral-specific plasmid construct.

One aspect of one embodiment relates to the disproportionately large ratio of the number of feeder cells used relative to the number of target T cells. For optimal growth of the T cells, the ratio of T cells:PBMC should be at least about 1:40 (i.e. at least about a 40-fold excess of PBMC), preferably the T cell:PBMC ratio is at least about 1:200, more preferably it is between about 1:400 and 1:800. Typically, we use a ratio of about 1:500.

The expansion can be even further enhanced by the inclusion of LCL feeder cells, preferably at a T cell:LCL ratio of at least about 1:10, more preferably at least about 1:20, still more preferably between about 1:50 and 1:200, most preferably about 1:100.

Use of feeder PBMC cells to assist in the culture of transduced host cells may be used if desired. Methods for the culture and production of feeder cells are known in the art and include for example, Garbrecht F. C. et al. J. Immunol. Methods 107:137-142, 1980; Riddell, J. Immunol. Meth. 128:189-201, 1990; and Londei M., et al., Scand J. Immunol. 27:35-46, 1988, each of which is incorporated by reference in their entirety. The addition of irradiated PBMC as feeder cells improves the ability of the T cells to enter a resting phase and to remain viable. Preferably, the ratio of PBMC feeder cells to resting T cells is at least about 2:1. Without the addition of PBMC feeder cells, viability of the T cells generally drops significantly (typically to levels of about 10% or less).

As described below, the T cells assume a small round morphology and 60-95% remain viable by trypan blue dye exclusion even after 28 days in culture. T cells propagated as such can also enter a resting phase upon IL-2 withdrawal; and they do not undergo programmed cell death (i.e. apoptosis) upon restimulation via the antigen-specific T cell receptor. Upon restimulation (e.g. with anti-CD3 mAb or antigen), the T cells reacquire responsiveness to IL-2, and can enter the S and G.sub.2 phases of the cell cycle and increase in cell number. Such characteristics are believed to be important for in vivo survival of the cells and for the efficacy of adoptive immunotherapy. In contrast, certain previously-described methods for the propagation of T cells have been reported to cause apoptotic cell death in a proportion of cells after cytokine withdrawal or T cell receptor restimulation (see, e.g, Boehme S A and Lenardo M J, Eur. J. Immunol., 23:1552-1560, 1992).

The T cell receptor activation signal (normally provided by antigen and antigen-presenting cells) may be augmented by the addition anti-CD3 monoclonal antibodies to the culture system. The anti-CD3 monoclonal antibody most commonly used is OKT3, which is commercially available from Ortho Pharmaceuticals in a formulation suitable for clinical use. The use of αCD3 mAb rather than antigen as a means of ligating the T cell receptor bypasses the need to have a source of antigen-presenting cells, which for virus-specific T cells would require maintaining large numbers of suitable autologous cells and infecting these cells in vitro with high titer virus. A concentration of anti-CD3 monoclonal antibody of at least about 0.5 ng/ml, preferably at least about 1 ng/ml, more preferably at least about 2 ng/ml, promotes the rapid expansion of the T cells such that a 500- to 3000-fold expansion can be achieved within about 10 to 13 days of growth using the methods. Typically, we use a concentration of about 30 ng/ml anti-CD3 monoclonal antibody. Although, as shown in FIG., much lower concentrations can also be used.

The culture media for use in the methods can be any of the commercially available media, preferably one containing: RPMI, 25 mM HEPES, 25 mu.M 2-mercaptoethanol, 4 mM L-glutamine, and 11% human AB serum. Fetal calf serum can be substituted for human AB serum. Preferably, after addition of irradiated feeder cells, anti-CD3 monoclonal antibody, and culture media are added to the target CTL or helper T cell, the mixture is allowed to incubate at 37° C. in a 5% CO₂ humidified atmosphere under standard cell culture conditions, which are well known in the art. Typically, such conditions may include venting; and addition of CO₂ if necessary (e.g., 5% CO₂, in a humidified incubator).

In addition, it is useful to include in the T cells a positive marker that enables the selection of cells of the negative selectable phenotype in vitro. The positive selectable marker may be a gene which, upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the aminoglycoside phosphotransferase gene (neo or aph) from Tn5 which codes for resistance to the antibiotic G418, the dihydrofolate reductase (DHFR) gene, the adenosine daminase gene (ADA), and the multi-drug resistance (MDR) gene.

Preferably, the positive selectable marker and the negative selectable element are linked such that loss of the negative selectable element necessarily also is accompanied by loss of the positive selectable marker. Even more preferably, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 11:3374-3378, 1991. In addition, in preferred embodiments, the polynucleotides encoding the chimeric receptors are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT/US91/08442 and PCT/US94/05601, by S. D. Lupton, describing the use of bifunctional selectable fusion genes derived from fusing a dominant positive selectable markers with negative selectable markers.

Preferred positive selectable markers are derived from genes selected from the group consisting of hph, neo, and gpt, and preferred negative selectable markers are derived from genes selected from the group consisting of cytosine deaminase, HSV-I TK, VZV TK, HPRT, APRT and gpt. Especially preferred markers are bifunctional selectable fusion genes wherein the positive selectable marker is derived from hph or neo, and the negative selectable marker is derived from cytosine deaminase or a TK gene.

A variety of methods can be employed for transducing T lymphocytes, as is well known in the art. Typically, we carry out retroviral transductions as follows: on day 1 after stimulation using REM as described herein, we provide the cells with 20-30 units/ml IL-2; on day 3, we replace one half of the medium with retroviral supernatant prepared according to standard methods and then supplement the cultures with 5 .mu.g/ml polybrene and 20-30 units/ml IL-2; on day 4, we wash the cells and place them in fresh culture medium supplemented with 20-30 units/ml IL-2; on day 5, we repeat the exposure to retrovirus; on day 6, we place the cells in selective medium (containing, e.g., an antibiotic corresponding to an antiobiotic resistance gene provided in the retroviral vector) supplemented with 30 units/ml IL-2; on day 13, we separate viable cells from dead cells using Ficoll Hypaque density gradient separation and then subclone the viable cells using the rapid expansion method described herein. Electrotransfer of linearized plasmids is accomplished by nucleofection carried out by standard electrophoresis procedures known in the art.

A flexible culturing system allows for the expansion and identification of T cells with other desired specificities. For example, autologous T cells can be genetically modified to express a fusion protein of hygromycin and pp65 (or any other desired viral antigen) in order to generate hygromycin-resistant T cells capable of expressing that antigen. These T cells can then be used to expand autologous antigen-specific T cells. Hygromycin-resistant MP1-specific T cells genetically modified to express the gene HyMP1 are capable of presenting the MP1 protein through the class I and II pathways to CD8+ and CD4+ T cells, respectively. Furthermore, a soluble fusion protein of CMV pp65 and IE can be processed by monocytes and used to expand CMV-specific T cells from PBMC. Expression of CCR7 in these cells provides the ability to traffic to secondary lymphoid tissue, which greatly enhances the anti-tumor effects of these cells. This may be provided naturally or by genetic modification.

To safeguard patient safety, non-immunogenic selection and suicide systems, such as dimerizable Fas, may be incorporated into the system. Also, to avoid initiating a hygromycin-specific immune response from antigen-presenting T cells expressing hygromycin phosphotransferase that would delete effector cells expressing HyTK gene, a fusion gene combining neomycin and MP1 may be used. Additional embodiments may include removal of immunogenic transgenes from the effector cells to reduce the possibility of immune-mediated elimination of the transferred T cells and inhibiting the expression of classical HLA molecules on bi-specific effector T cells to prevent antigen recognition by T cells in a recipient of adoptive immunotherapy. Antigen presentation capacity of T cells also may be improved by co-expressing additional T cell co-stimulatory molecules such as found on professional antigen presenting cells. Generation of fusion genes does not rely on partnering the viral antigen with hygromycin. Other antibiotic-resistance genes can be used, such as neomycin phosphotransferase.

MP1-specific T cells can be generated, for example, by obtaining PBMC from an influenza sero-positive normal volunteer donor that contains about 1% MP1-tetramer+ CD8+ circulating T cells. Endogenous influenza MP1-specific specific T cells can be expanded from these cells using repetitive 7-day stimulation cycles with irradiated hygromycin-resistant autologous T cells genetically modified to express the fusion protein hygromycin::MP1 (HyMP1). These PBMC may be incubated with irradiated MP1-presenting T cells (PBMC:T cellŝHyMP1+) at a ratio of about 1:1 to 10:1 in the presence of low-dose (about 5 U/mL) IL-2.

Following weekly stimulations with stimulating T cells, a large population of MP1-tetramer+ population of MP1-specific (tetramer+) T cells emerges in the culture and can be isolated easily using methods known in the art. For example, PBMC from an HLA-A2+ volunteer donor initially containing about 1% MP1-tetramer+ CD8+ circulating T cells, were incubated at a 5:1 ratio (PBMC:T cellŝHYMP1+) in the presence of 5 U/mL IL-2. After 21 days of repetitive in vitro stimulations the percentage of MP1-tetramer+ CD8+ T cells increased to about 50%, demonstrating that the HyMI fusion protein is processed through the MHC class I pathway and the immunoreactive GILGFVFTL peptide (SEQ ID NO:3) can be presented by autologous T cells. In addition to CD8+ MP1-tetramer+ T cells, the culture conditions also expanded CD8+ MP1-tetramer+ T cells and CD4+ T cells. A ready supply (>10̂9) of HyMP1+ stimulator T cells can be maintained using repetitive OKT3-driven expansion cycles, growing in the presence of cytocidal concentrations of hygromycin (0.2 mg/mL). The stimulator T cells grown in this fashion have been characterized as CD8+ CD80+HLA-ABC+HLA-DR+MP1-tetramer—as assessed by flow cytometry.

Alternatively, the PBMC may be repetitively incubated with soluble MP1 protein. The soluble protein is taken up and processed by the MHC machinery of monocytes, presenting the antigen and resulting in stimulation and preferential expansion of MP1-specific T cells. These MP1-specific cells then can be isolated using conventional methods, such as magnetic bead separation, based on production of IFN-γ and their specificity for MP1 again verified.

Non-human primate and human T cells that have been genetically modified to express immunogenic proteins according to one embodiment are capable of antigen delivery and trafficking to lymph nodes in vivo after intravenous administration, as demonstrated in the examples appended below. These data demonstrate that autologous T cells act as antigen-presenting cells to stimulate a recall response in vitro against the viral antigen MP1, and that the expanded MP1-specific T cells can be rendered specific for CD19. In addition, both the endogenous MP1-specific and introduced CD19-specific immunoreceptors can activate genetically modified T cells independently. The sequential and/or simultaneous engagement of both immunoreceptors results in augmented activation of the effector cells which translates into improved potency by combining autologous MP1+ antigen-presenting T cells with MP1-tetramer+Fc+ T cells for treating established CD19+ tumors in vivo. Trafficking to lymphoid tissue allows highly stimulated tumor-specific T cells to produce their effect in the absence of a physiologic CD4+ helper-response. The in vivo persistence of adoptively transferred CTL may be maintained with exogenous IL-2.

Methods for production of APCs. An in vitro system for generating antigen-presenting cells that can be used for immunization or generation of viral-specific T cells is also provided. T cells or other PBMC may be genetically modified to express, for example, a chimeric protein of hygromycin (Hy) phosphotransferase fused to the influenza A matrix protein 1 (MP1). The fusion protein confers resistance to hygromycin, permitting in vitro selection of genetically modified cells, while the MP1-portion is processed through the T cell proteosome apparatus. Using PBMC from an HLA-A2+ donor, CD8+ MP1-tetramer+ T cells could be rapidly expanded by co-culture with irradiated autologous MP1+Hy+antigen-presenting T cells. Specificity of the expanded T cells for MP1 was demonstrated by secretion of IFN-γ upon co-culture with HLA-restricted cells expressing MP1. The influenza-specific T cells then were rendered bi-specific by introduction of a chimeric immunoreceptor specific for the CD19 determinant, termed CD19R. This chimeric immunoreceptor molecule can dock with the CD19 determinant through an extracellular domain, derived from the scFv of a CD19-specific mouse mAb, leading to T cell activation through the attached CD3-.zeta. chain (Cooper et al., Blood 101 (4):1637-1644, 2002). Bi-specificity was demonstrated by chromium release assays in which the MP1-tetramer+CD19R+ T cells lysed both MP1+ and CD19R+ targets. Conversely, monospecific MP1-tetramer+ T cells and CD19R+ T cells killed only MP1+ or CD19+ targets, respectively. Bi-specific MP1-tetramer+CD19+CD8+ T cells could lyse autologous targets expressing MP1 as well as targets expressing a CD19 determinant, whereas CD19+CD8+ T cells could only lyse CD19+ targets. The specificity for cognate antigen was demonstrated by the fact that neither effector T cell could lyse autologous T cells.

The technique of using hygromycin fusion proteins to present MP1 can be applied to other viral antigens as well. For example, fusion molecules may be constructed using a modified CMV pp65 gene combined with hygromycin phosphotransferase, designated as Hypp65. pp65 cDNA may be modified to decrease the innate protein kinase activity that is toxic to cells expressing this protein It has also been demonstrated that pp65 can be expressed in human cells grown under cytocidal concentrations of hygromycin. Cells growing in 1.6 mg/mL hygromycin B were plated onto glass slides, fixed, permeabolized and stained with mouse anti-CMV mAb using reagents and protocols from Biotest Diagnostics Corporation.

Immunoreactive pp65 proteins are presented through the MHC class I pathway since pp65-tetramer+ CD8+ T cell clones from a HLA A2+ CMV sero-positive donor are able to lyse HLA A2+ cells genetically modified with a plasmid expressing Hypp65. Controls include hygromycin-resistant U293T cells electroporated with the pMG plasmid incubated with and without the CMV pp65 peptide NLVPMVATV (SEQ ID NO:1). T2 cells are HLA A2+ T-B lymphoblast hybrids incubated with and without the CMV pp65 peptide. These same methods may be used with any viral antigen.

In adoptive immunotherapy, one or more specific immunities can be conferred upon an individual by transferring T cells having the desired antigenic specificities. The cells of interest may be derived from the immunodeficient host or from a compatible specifically immunized host. The latter source is of course especially important in situations in which the immunodeficient host has an insufficient number of T cells, or has T cells that are insufficiently effective.

As used herein, “adoptive immunotherapy” refers to administration of donor or autologous T lymphocytes for the treatment of a disease or disease condition wherein the disease or disease condition may coincide with an insufficient or inadequate immune response.

In order to augment or reconstitute T cell responses in such immunodeficient hosts, the antigen-specific T cells must be grown to large numbers in vitro and then administered intravenously to the immune-deficient host. After undergoing adoptive immunotherapy, hosts that previously had inadequate or absent responses to antigens expressed by pathogens or tumors, may express sufficient immune responses to become resistant or immune to the pathogen or tumor.

For treatment of human disease, the use in immunotherapy of cloned antigen-specific T cells, which represent the progeny of single cells, offers significant advantages because the specificity and function of these cells can be rigorously defined and precise dose:response effects evaluated. Riddell et al. were the first to adoptively transfer human antigen-specific T cell clones to restore deficient immunity in humans. Riddell, S. R. et al., “Restoration of Viral Immunity in Immunodeficient Humans by the Adoptive Transfer of T Cell Clones”, Science 257:238-240 (1992

In a study, Riddell et al. used adoptive immunotherapy to restore deficient immunity to cytomegalovirus in allogeneic bone marrow transplant recipients. Cytomegalovirus specific CD8+ cytotoxic T cell clones were isolated from three CMV seropositive bone marrow donors, propagated in vitro for 5 to 12 weeks to achieve numerical expansion of effector T cells, and then administered intravenously to the respective bone marrow transplant (BMT) recipients. The BMT recipients were deficient in CMV-specific immunity due to ablation of host T cell responses by the pretransplant chemoradiotherapy and the delay in recovery of donor immunity commonly observed after allogeneic bone marrow transplant (Reusser et al. Blood, 78:1373-1380, 1991). Riddell et al. found that no toxicity was encountered and that the transferred T cell clones provided these immunodeficient hosts with rapid and persistent reconstitution of CD8+ cytomegalovirus-specific CTL responses.

One exemplary method for isolating and culturing the CD8+ CMV-specific T cell clones includes: peripheral blood mononuclear cells (PBMCs) derived from the bone marrow donor were first cultured with autologous cytomegalovirus-infected fibroblasts to activate CMV-specific CTL precursors. Cultured T cells were then restimulated with CMV-infected fibroblasts and the cultures supplemented with gamma-irradiated (γ-irradiated) PBMCs. 2-5 U/ml of interleukin-2 (IL-2) in suitable culture media was added on days 2 and 4 after restimulation to promote expansion of CD8+ CTL (Riddell et al., J. Immunol., 146:2795-2804, 1991) which is hereby incorporated by reference. To isolate T cell clones, the polyclonal CD8+ CMV-specific T cells were plated at limiting dilution (0.3-0.6 cells/well) in 96-well round bottom wells with either CMV-infected fibroblasts as antigen-presenting cells (Riddell, J. Immunol., 146:2795-2804, 1991); or aCD3 monoclonal antibody to mimic the stimulus provided by antigen-presenting cells. (Riddell, J. Imm. Methods, 128:189-201, 1990). Then, y-irradiated peripheral blood mononuclear cells (PBMC) and EBV-transformed lymphoblastoid cell line (LCL) were added to the microwells as feeder cells. Wells positive for clonal T cell growth were evident in 10-14 days. The clonally derived cells were then propagated to large numbers initially in 48 or 24 inch plates and subsequently in 12-well plates or 75-cmA2 tissue culture flasks. T cell growth was promoted by restimulation every 7-10 days with autologous CMV-infected fibroblasts and γ-irradiated feeder cells consisting of PBMC and LCL, and the addition of 25-50 U/ml of IL-2 at 2 and 4 days after restimulation.

Antigen-specific T cells or APC as method of treatment. Another embodiment is directed toward a method of treatment for opportunistic viral infections in immunosuppressed individuals or other individuals who have contracted the virus or viruses wherein autologous T cells modified with a viral-specific fusion transgene such as dual chimeric antigen or TriVi can be clinically infused as a vaccine to expand T cells in vivo against desired viral epitopes. The method of treatment comprising clinical infusion of viral-specific T cells may be administered prior to primary infection or for reactivation of infection or for acute treatment of a primary or reactivated infection. The modified T cells are preferably generated from Tcm cells in order to confer persistent immunity to immunosuppressed individuals. Antiviral-specific cell-mediated immune reconstitution can also be achieved using the antigen-specific T cells or APC methods provided.

In another aspect, dual or TriVi specific PBMCs or Tcm cells may be used to reestablish or augment the immune response to primary or reactivated infection caused by EBV, CMV, Influenza A, or any combination of the three viruses in an immunosuppressed individual. The cause of the immunosuppression may be stem cell, bone marrow, or solid organ transplant patients, HIV, or other conditions wherein the immune system has been compromised.

The lymphocytes may be used to confer immunity to individuals. By “immunity” is meant a lessening of one or more physical symptoms associated with a response to infection by a pathogen, or to a tumor, to which the lymphocyte response is directed. The amount of cells administered is usually in the range present in normal individuals with immunity to the pathogen. Thus, CD8+ CD4− cells are usually administered by infusion, with each infusion in a range of at least 10̂6 to 10̂10 cells/m̂2, preferably in the range of at least 10̂7 to 10̂9 cells/m̂2. The clones may be administered by a single infusion, or by multiple infusions over a range of time. However, since different individuals are expected to vary in responsiveness, the type and amount of cells infused, as well as the number of infusions and the time range over which multiple infusions are given are determined by the attending physician, and can be determined by routine examination. The generation of sufficient levels of T lymphocytes having preselected or desired viral specificity (including cytotoxic T lymphcytes and/or helper T lymphocytes) is readily achievable using a rapid expansion method (Riddell et al., U.S. Pat. No. 5,827,642) and the vectors, and methods as disclosed herein.

It has also been observed that T cells expanded using rapid expansion methodology exhibit very high levels of transduction using vectors such as retroviral vectors which will be of great use in the contexts of adoptive immunotherapy and gene therapy using lymphocytes. The genetic transduction of rapidly-expanded T cells and the use of such genetically-transduced antigen-specific CTLs for adoptive immunotherapy in human patients is described, for example, in U.S. Pat. No. 5,827,642 (hereby incorporated by reference in its entirety).

The strategy of isolating and expanding antigen-specific T cells as a therapeutic intervention for human disease has been validated in clinical trials. Riddell et al., Science 257:238, 1992; Walter et al., N. Engl. J. Med. 333:1038, 1995; Heslop et al., Nat. Med. 2:551, 1996. Initial studies have evaluated the utility of adoptive T cell therapy with CD8+ cytolytic T cell (CTL) clones specific for cytomegalovirus-encoded antigens as a means of reconstituting deficient viral immunity in the setting of allogeneic bone marrow transplantation and have defined the principles and methodologies for T cell isolation, cloning, expansion and re-infusion (Riddell et al., supra). A similar approach has been taken for controlling post-transplant EBV-associated lymphoproliferative disease. EBV-specific donor-derived T cells have the capacity to protect patients at high risk for this complication as well as eradicate clinically evident disease which mimics immunoblastic B cell lymphoma (Heslop et al., supra). These studies clearly demonstrate that adoptively transferred ex vivo expanded T cells can mediate antigen-specific effector functions with minimal toxicities and have been facilitated by targeting defined virally-encoded antigens to which T cell donors have established immunity.

Conventional techniques of molecular biology, microbiology, cell biology, recombinant DNA, and immunology are employed, which are within the skill of the art. Such techniques are explained fully in the literature. See e.g., Sambrook, Fritsch, and Maniatis, Molecular Cloning: A Laboratory Manual, Second Edition (1989), Oligonucleotide Synthesis (M. J. Gait Ed., 1984), Animal Cell Culture (R. I. Freshney, Ed., 1987), the series Methods in Enzymology (Academic Press, Inc.); Gene Transfer Vectors for Mammalian Cells (J. M. Miller and M. P. Calos eds. 1987), Handbook of Experimental Immunology, (D. M. Weir and C. C. Blackwell, Eds.), Current Protocols in Molecular Biology (F. M. Ausubel, R. Brent, R. E. Kingston, D. D. Moore, J. G. Siedman, J. A. Smith, and K. Struhl, eds., 1987), and Current Protocols in Immunology (J. E. Coligan, A. M. Kruisbeek, D. H. Margulies, E. M. Shevach and W. Strober, eds., 1991). All patents, patent applications, and publications mentioned herein, both supra and infra, are hereby incorporated herein by reference.

The following examples are provided to better illustrate the embodiments and are not to be interpreted as limiting the scope of any claimed embodiment. The extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

### TriVi Plasmid Construct

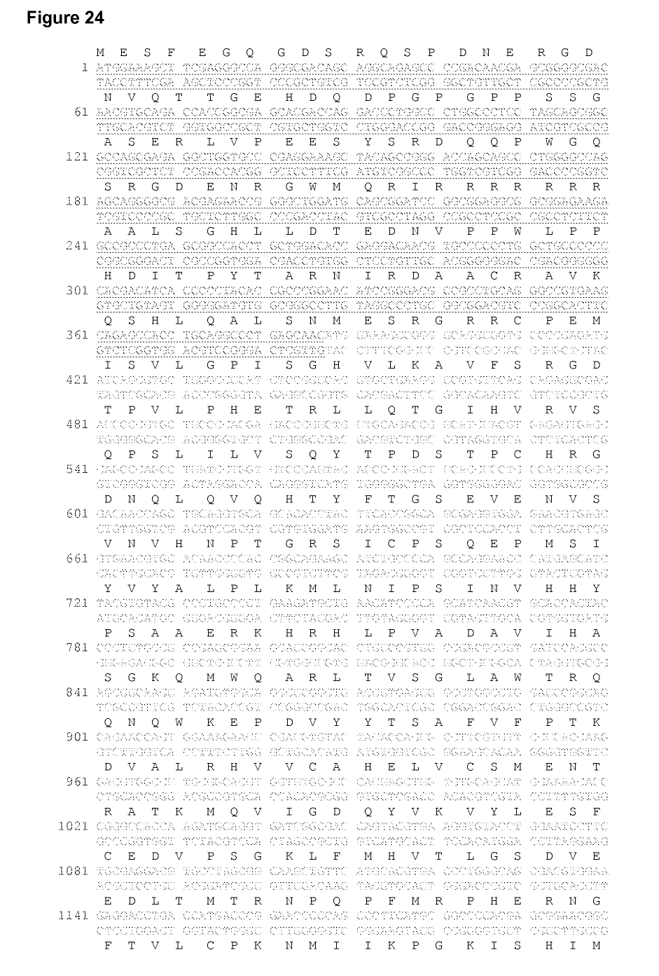

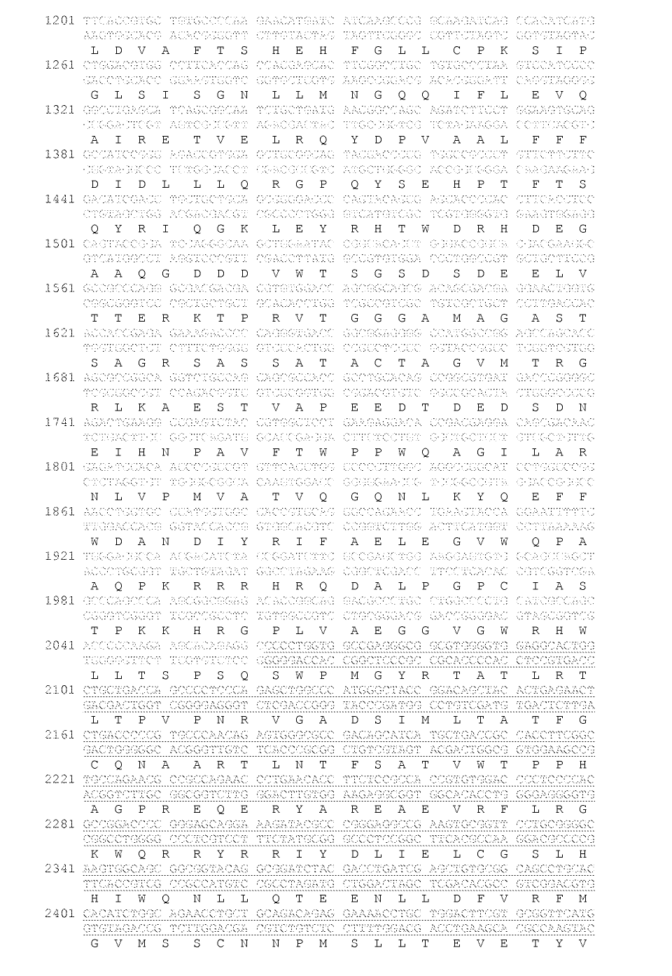

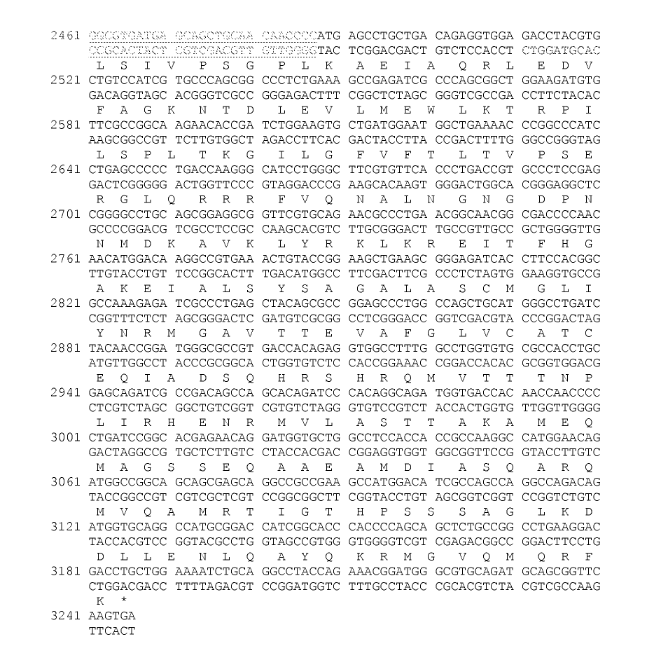

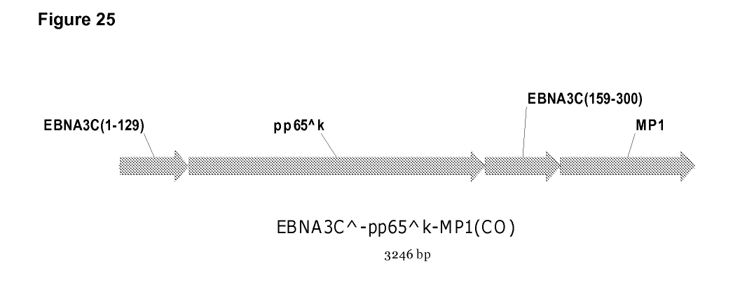

One exemplary TriVi construct (EBNA3Ĉpp65̂k-MP1) was designed de novo for the simultaneous expression of a single polypeptide that contains domains of CMV pp65, EBV EBNA3C, and Influenza A MP1. The codon optimized cDNA consists of pp65 modified to eliminate protein kinase activity by deletion of amino acids 436-438 flanked by amino acids 1-129 of EBNA3C on its N-terminal and amino acids 159-300 of EBNA3C on its C-terminal. Amino Acids 130-158 of EBNA3C, which are implicated in perturbation of cell cycle control, are eliminated. To this chimera, Influenza A MP1 was fused to the C-terminal, resulting in the full length TriVi Construct. The DNA and protein sequences of the TriVi construct can be seen in FIG. 24. SEQ ID NO: 7 is the coding strand of TriVi DNA. SEQ ID NO: 8 is the complementary strand of TriVi DNA. SEQ ID NO: 9 is the TriVi amino acid sequence.

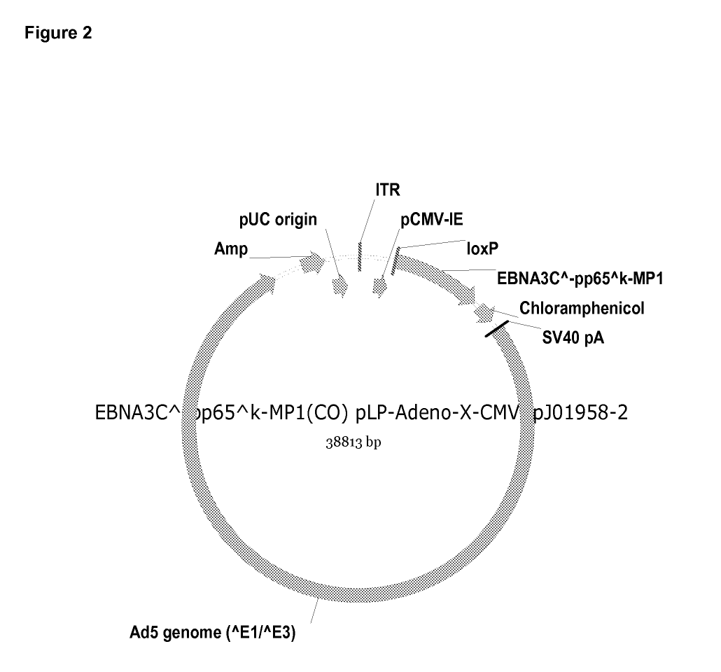

The TriVi construct is inserted at a loxP site into a recombinant adenovirus vector that contains a ΔE1/ΔE3 replication-deficient, type 5 adenovirus genome (Ad5) which has been engineered for use in gene delivery and expression studies. The loxP site is located just downstream of the immediate early promoter of human CMV (PCMV-IE). There is a bacterial promoter to drive expression of the chloramphenicol resistance gene that is transferred from the Creator Donor Vector (pDNR-CMV). Inverted terminal repeats (ITR) are necessary for the replication of adenoviral DNA and is exposed by digestion with PacI. The adenovirus vector also contains a pUC replication origin and an ampicillin resistance gene (Amp) for propagation and selection. The plasmid map of the resulting recombinant adenovirus (EBNA3Ĉpp65̂k-MP1 (CO) pLP-Adeno-X-CMV; pJ01958-2) can be seen in FIG. 2. Other vectors may be used to create the TriVi plasmid construct, including the chimeric adenovirus, Ad5/F35, and others discussed herein.

### Example 2

### TriVi Plasmid Construct is Transfected into the Nucleus of Target Cells by Electroporation

For non-viral gene transfection, two micrograms of linearized TriVi plasmid was premixed in lipofectamine and gently dispersed onto 296T cells in 6-well tissue culture plates. Transfection of 296T cells was achieved using a single pulse of 240 V for 40 psec in a Multiporator device with 10 μg linearized TriVi plasmid in hypo-osmolar buffer.

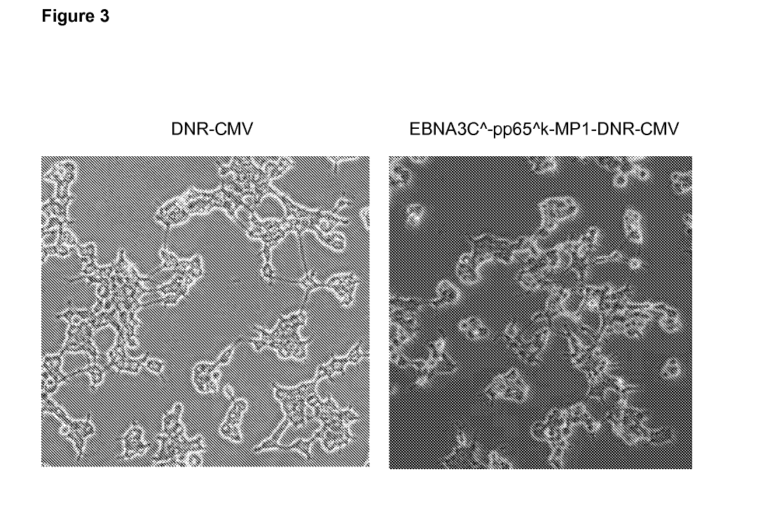

Successful transfer of the linearized TriVi plasmid was measured by pp65 immunochemical staining for the EBNA3Ĉpp65̂k-MP1 gene in 293T cells using the ABC (Avidin and Biotinylated horseradish peroxidase macromolecular Complex) technique. Briefly, the transfected 293T cells were fixed and washed, then stained with primary CMVpp65 antibody, followed by secondary biotinylated antibody and then a preformed avidin and biotinylated horseradish peroxidase macromolecular complex. After incubating with hydrogen peroxide, the EBNA3Ĉpp65̂k-MP1 gene is visualized with the peroxidase substrate 3-amino-9-3thylcarbazole (AEC), which stains the nucleus red. FIG. 3 shows positive pp65 staining for the EBNA3Ĉpp65̂k-MP1 gene (right) as compared to an empty DNR-CMV donor vector lacking the EBNA3Ĉpp65̂k-MP1 gene (left).

### Example 3

### Western Blot Confirms Transfection of TriVi Plasmid Construct Results in Expression of TriVi Antigens

Western analyses were performed as follows. Twenty million transfected 293T cells were lysed on ice in 1 ml of RIPA buffer (PBS, 1% NP40, 0.5% sodium deoxycholate, 0.1% SDS) containing 1 tablet/10 ml Complete Protease Inhibitor Cocktail (Boehringer Mannheim, Penzberg, Federal Republic of Germany.). After 60 minutes, aliquots of centrifuged supernatant were boiled in an equal volume of loading buffer under reducing conditions and then subjected to SDS-PAGE electrophoresis on precast 12% acrylamide gels (Bio-Rad Laboratories, Hercules, Calif.).

Following transfer to nitrocellulose, membranes were blocked for 2 hours in Blotto solution containing 0.07 gm/ml non-fat dried milk. Membranes were washed in T-TBS (0.05% Tween 20 in Tris buffered saline, pH 8.0.) and incubated for 2 hours with influenza A MP1 or CMV pp65 antibodies. After washing in T-TBS and then developed with 30 ml of AKP solution (Promega, Madison, Wis.) according to manufacturer's instructions. The chemiluminescence was measured over a 2 hour period.

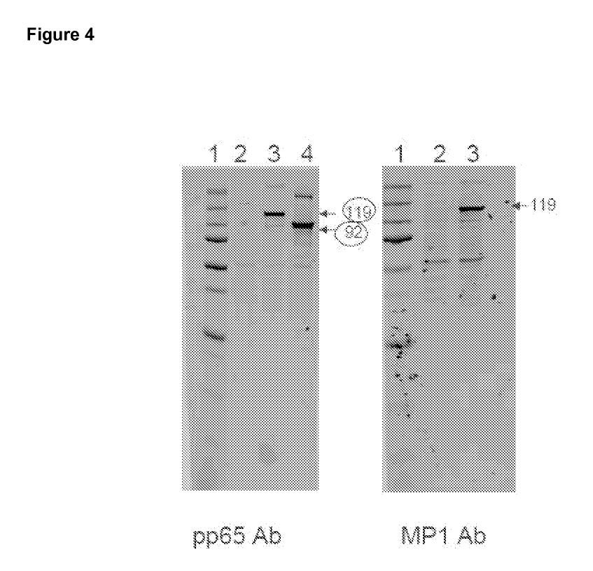

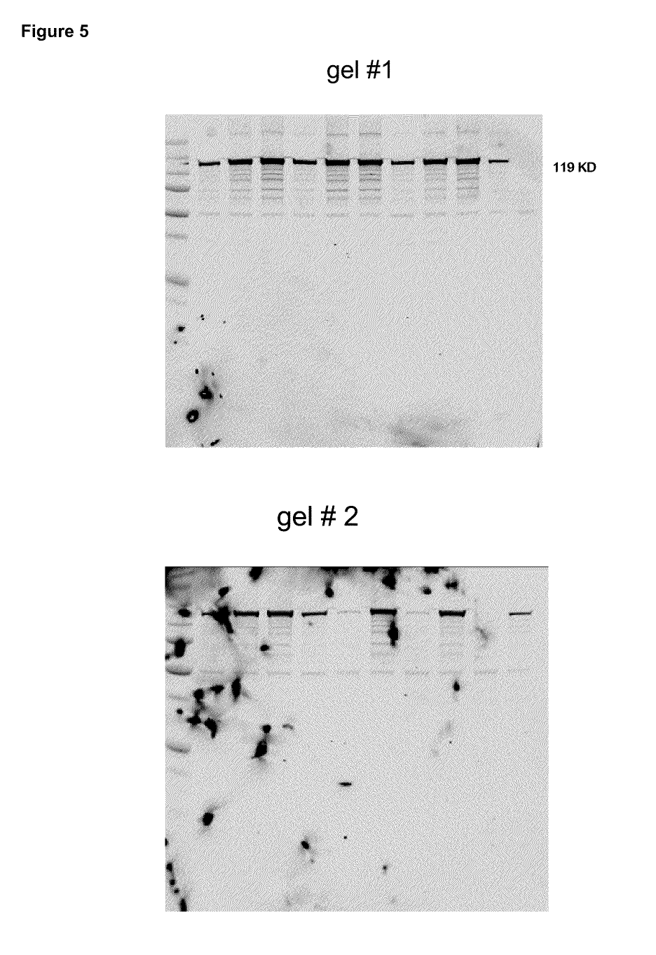

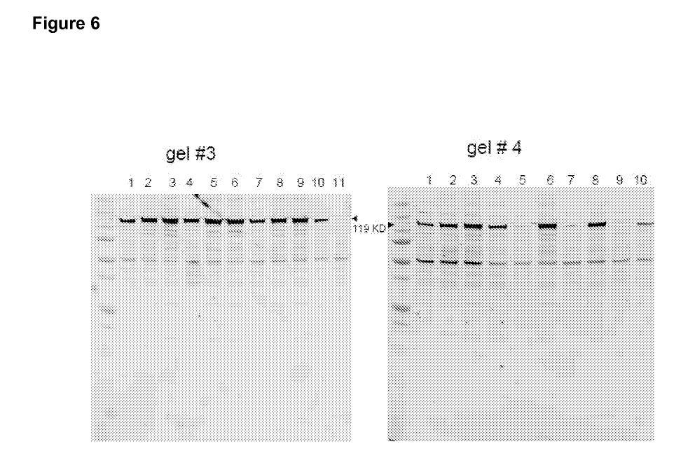

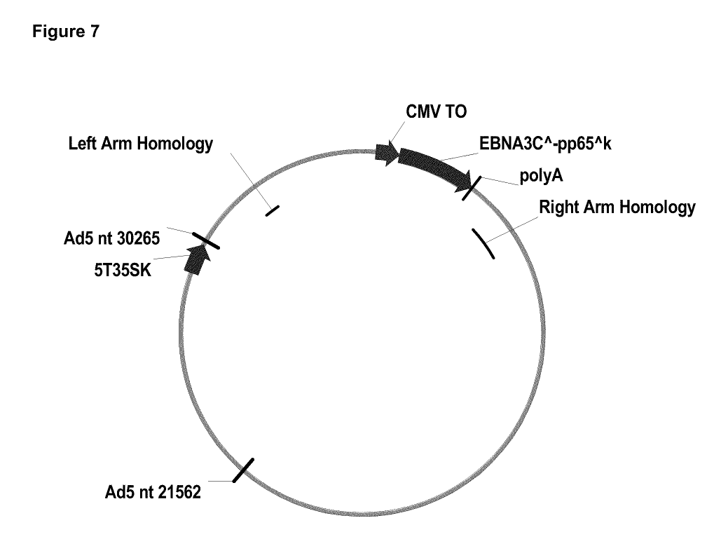

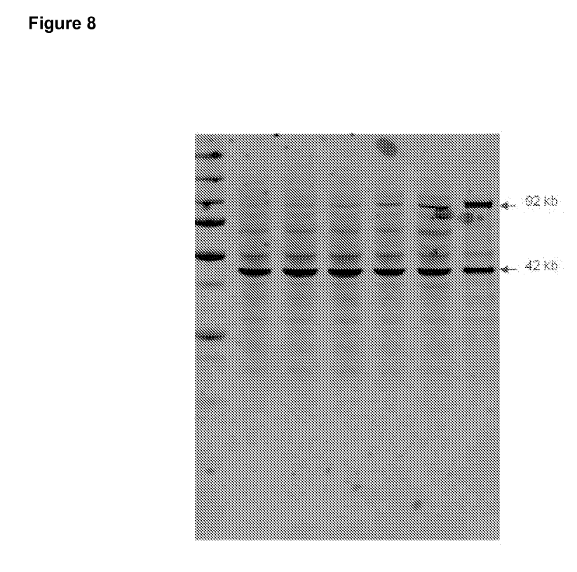

Western blot analysis as shown in FIG. 4 showed that 293T cells transfected with epJ01741(EBNA3Ĉ-pp65_pEK) and TriVi pJ01942 (EBNA3Ĉ-pp65̂k-MP1_pDNR-CMV) genes express pp65 as shown by corresponding expected molecular weight bands at 92 kD (lane 4, left side) and 119 kD (lane 3, left side), respectively. TriVi pJ01942 (EBNA3Ĉ-pp65̂k-MP1_pDNR-CMV) infected 293T cells also express MP1 (lane 3, right side). MP1 and pp65 were not present in control cells containing pJ01950#1 (pDNR-CMV) (lane 2, left and right sides).

### Example 4

### Generation of TriVi-Specific Viral Antigen Presenting Cells (v-APCs) and Successful generation of viral specific CD8+ T cells using v-APCs

Viral antigen presenting cells (v-APCs) were generated by transducing donor derived monocytes with linearized adenoviral vector Ad5/F35 containing the TriVi construct described above (pj01869). Transfection of donor monocytes was achieved three days after stimulation with 30 ng/mL of OKT3 by electroporating with a single pulse of 250 V for 40 μsec using a Multiporator device with 10 μg of linearized TriVi plasmid in hypo-osmolar buffer. Following a 10-minute incubation at room temperature unfractionated autologous PBMCs are washed and co-cultured in T-75 flasks with T cell growth media (RPMI 1640 supplemented with 25 mM HEPES and 10% FCS) containing OKT3, irradiated v-APC and irradiated LCL. IL-2 at 25 U/ml is added every week beginning 24 hours after electroporation. After two weeks, the resultant cells were expanded with conventional rapid expansion medium (REM) containing irradiated PBMC, irradiated LCL and OKT3.

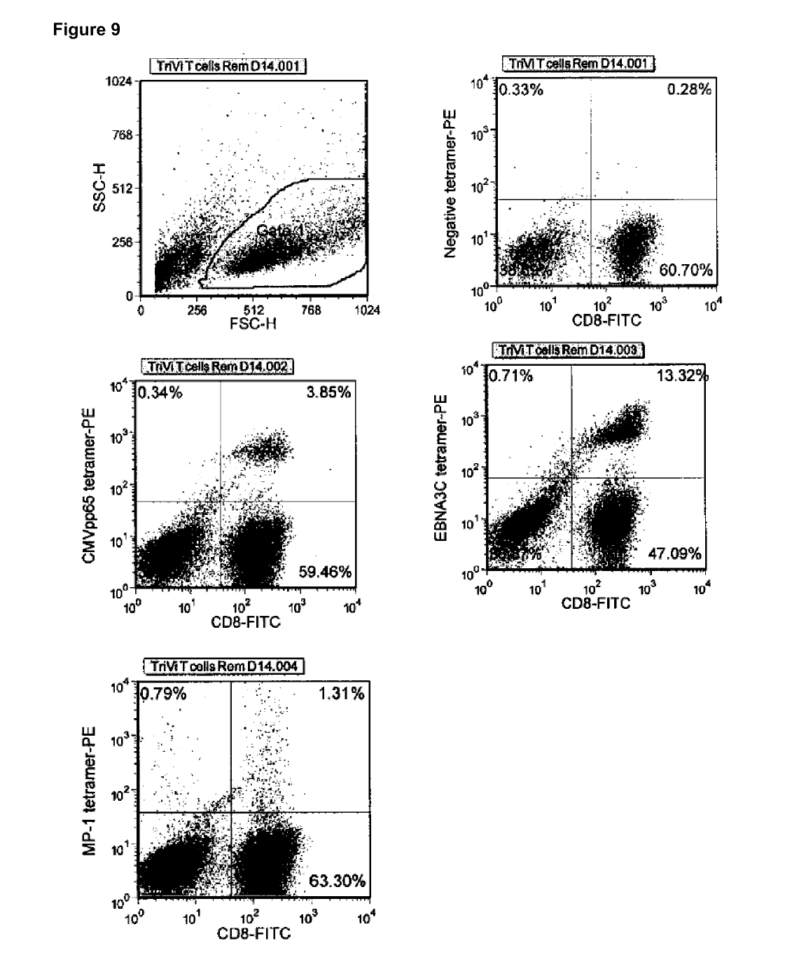

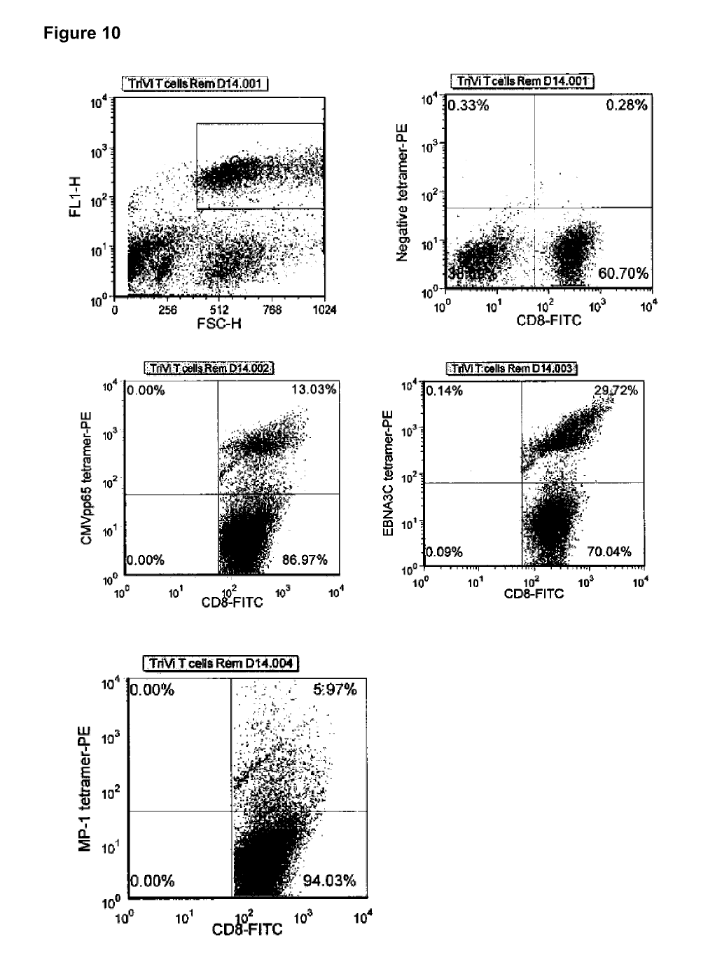

T cells that are specific for different viral antigens were analyzed by flow cytometry. Fluorescein isothiocyanate (FITC)-conjugated anti-CD8 antibody, and phycoerythrin (PE)-conjugated tetramers that specifically label T cells that express T cell receptors that are specific for the following peptide-MHC complex: Multi-allele negative tetramer, CMVpp65/HLA-A2 tetramer having the amino acid sequence NLVPMVATV (SEQ ID NO: 1), EBNA3C/HLA-A2 tetramer having the amino acid sequence LLDFVRFMGV (SEQ ID NO: 2), and MP1/HLA-A2 tetramer having the amino acid sequence GILGFVFTL (SEQ ID NO: 3). Scatter plots were generated to quantitate the number of CD8+ T cells specific to each viral peptide associated with v-APC MHC complexes as shown in FIG. 9.

### Example 5

### Simultaneous Expansion of CMV and EBV Specific T Cells using Chimeric CMVpp65: ENMA3C Transgene Expressing Peripheral Blood Antigen Presenting Cells

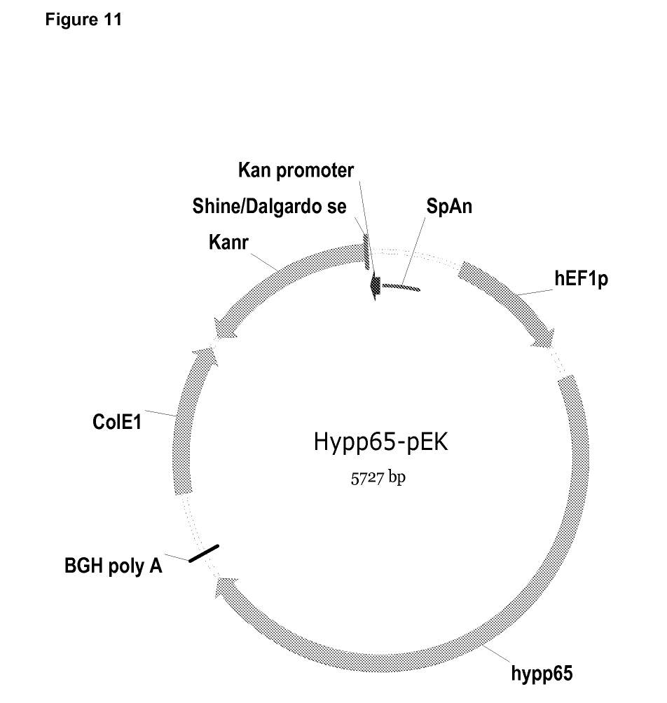

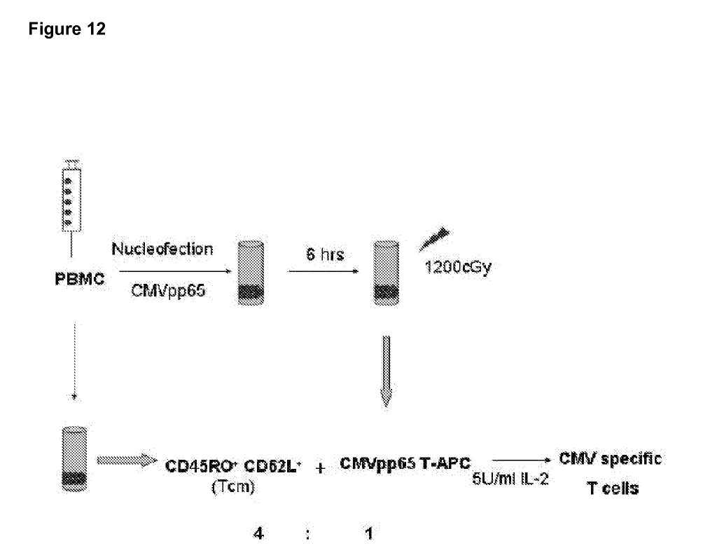

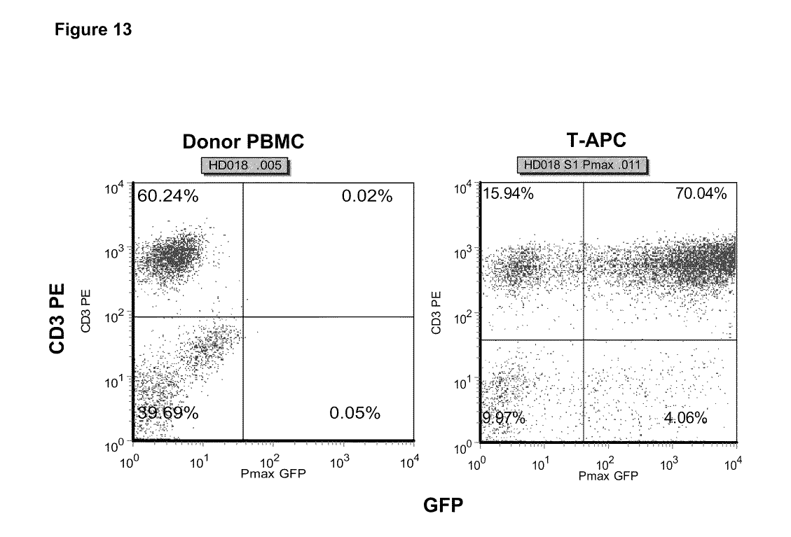

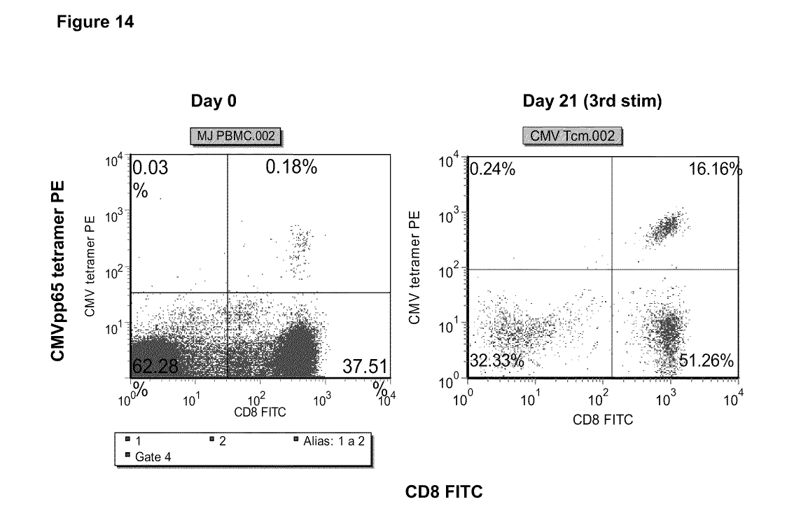

As shown by FIG. 12, viral antigen transgene⁺ APCs were generated by electrotransfer of our Hypp65-pEK plasmid that expresses the viral antigen cDNA from an EF-1 alpha promoter (FIG. 11) into unfractionated PBMC. Purified central memory enriched T cells (CD45RO⁺CD62L⁺) were co-cultured with irradiated APC at a 4:1 responder to stimulator ratio in the presence of 5 U/ml 1 L-2. These cultures were restimulated weekly.

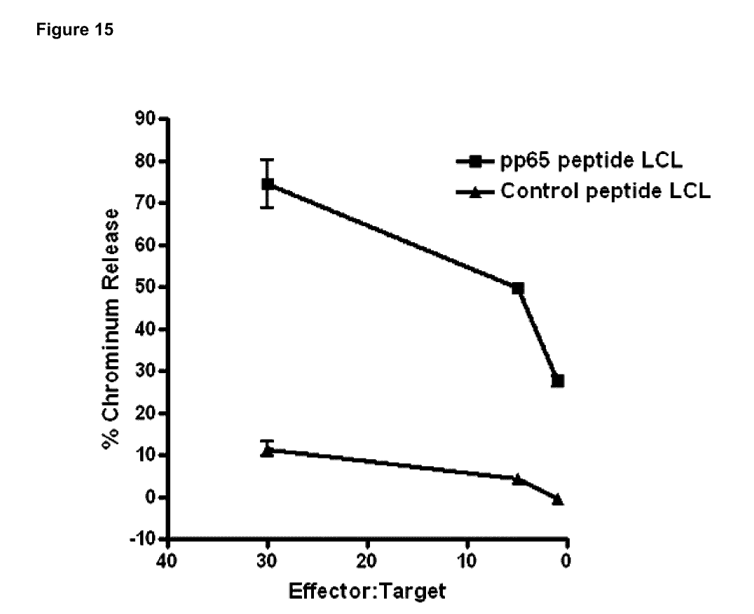

After three weeks, cultured cells expanded 3 fold, consisted of 30% CD4⁺ and 70% CD8 T cells. CMVpp65/HLA-A2 tetramer⁺ CD8 T cells increased from 0.5% to 32%. Cytotoxicity assays demonstrated the specific killing of CMVpp65 peptide loaded target cells by these T cell lines (FIG. 15).

### Example 6

### Generation of T Cell Responses to Both CMV and EBV in Seronegative Donor PBMC

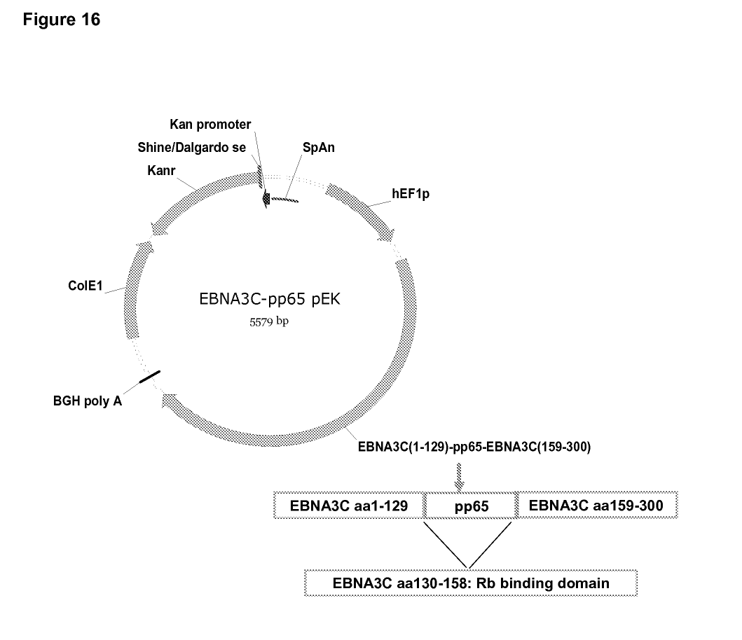

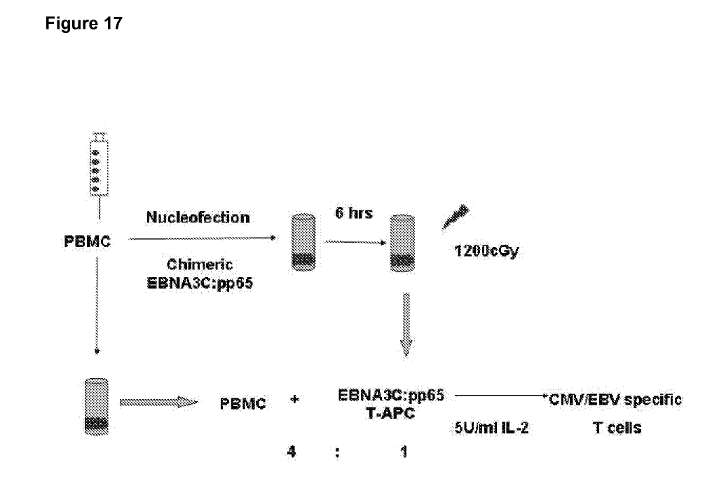

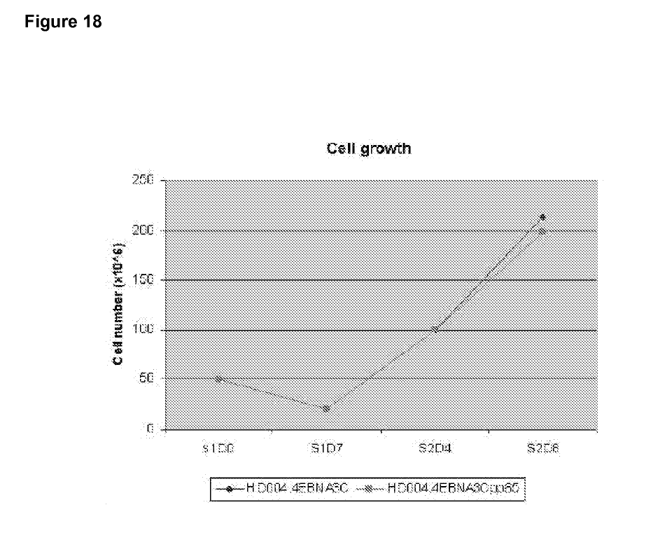

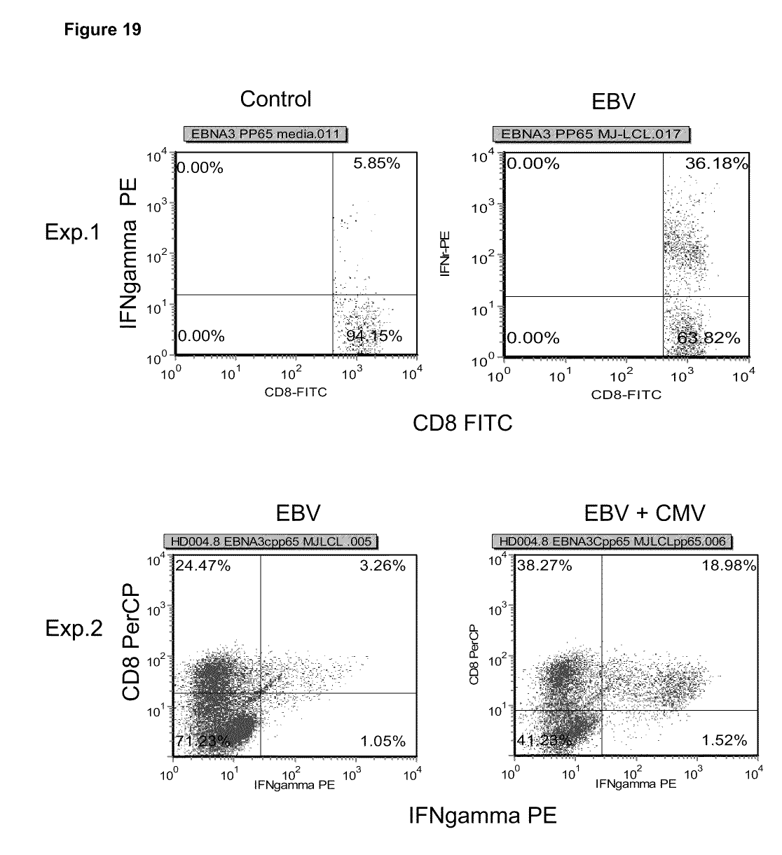

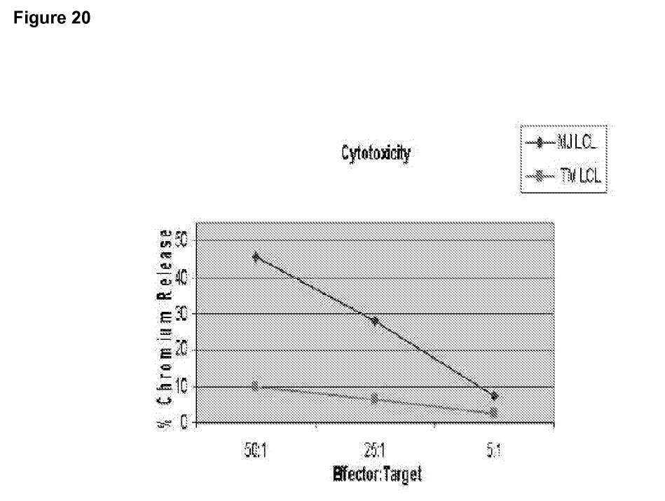

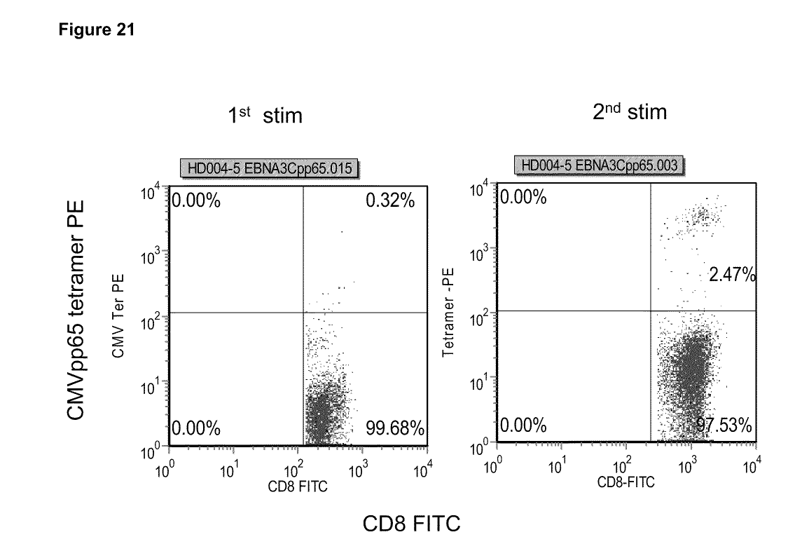

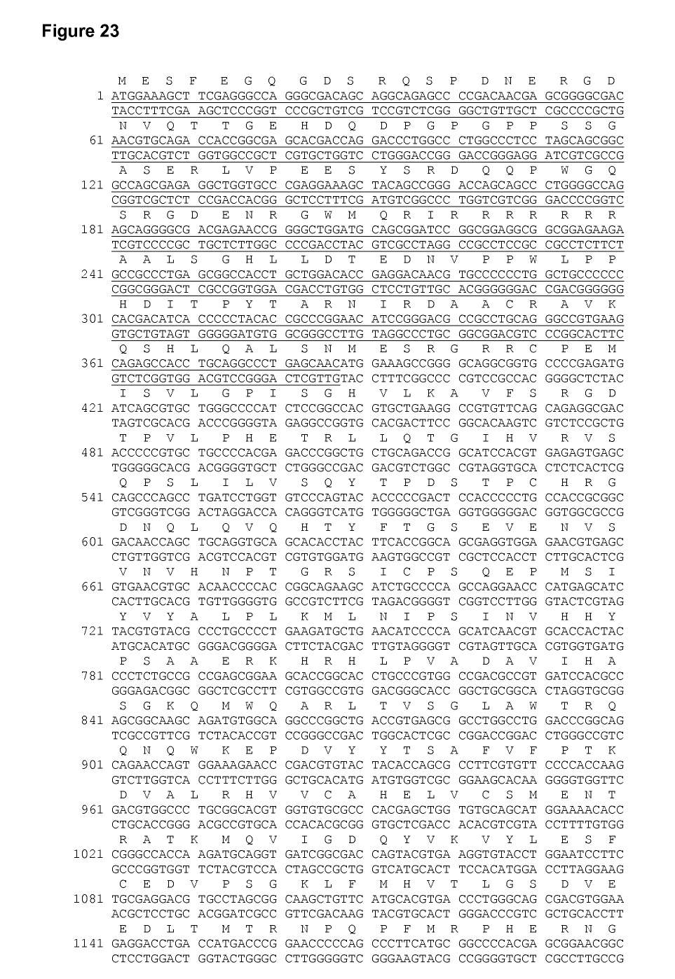

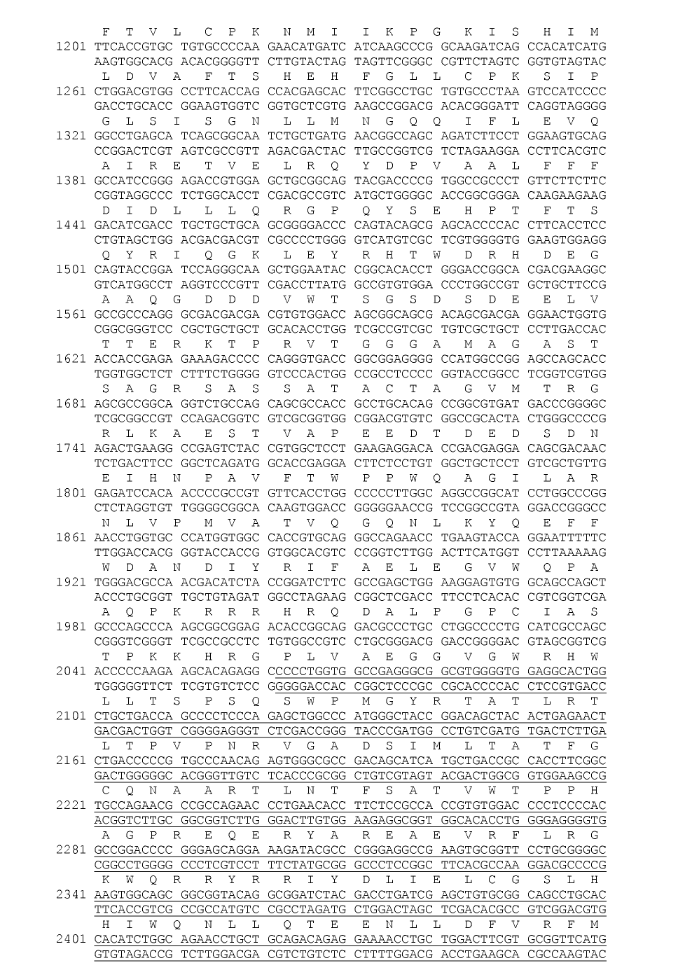

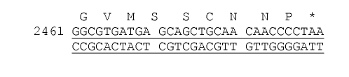

The potential to generate T cell responses to both CMV and EBV by expressing a pp65/EBNA3C fusion viral antigen transgene (FIG. 23) in PBMC was further investigated. Using healthy donors who were both CMV and EBV immune, cultures consisting of both CD4⁺ and CD8⁺ T cells having dual reactivity to both pp65 and EBNA3C were generated. As shown by FIG. 17, viral antigen transgene⁺ APCs were generated by nucleofection of our EBNA3C-pp65-pEK plasmid that expresses the viral antigen cDNA from an EF-1 alpha promoter (FIG. 16) into unfractionated PBMC by standard electroporation procedures. PBMC were co-cultured with irradiated EBNA3C:pp65 APC at a 4:1 responder to stimulator ratio in the presence of 5 U/ml 1 L-2. These cultures were restimulated weekly. After the second stimulation, a marked increase in both CD4 and CD8 cells was observed as shown in FIG. 18. EBV/CMV-specific T cells were generated as evidenced by CMVpp65 and EBNA3C/HLA-2 tetramer analysis and functional analysis using 4-hr chromium release assays and flow cytometric analysis for intracellular IFN-γ production were generated. See FIGS. 19-21.

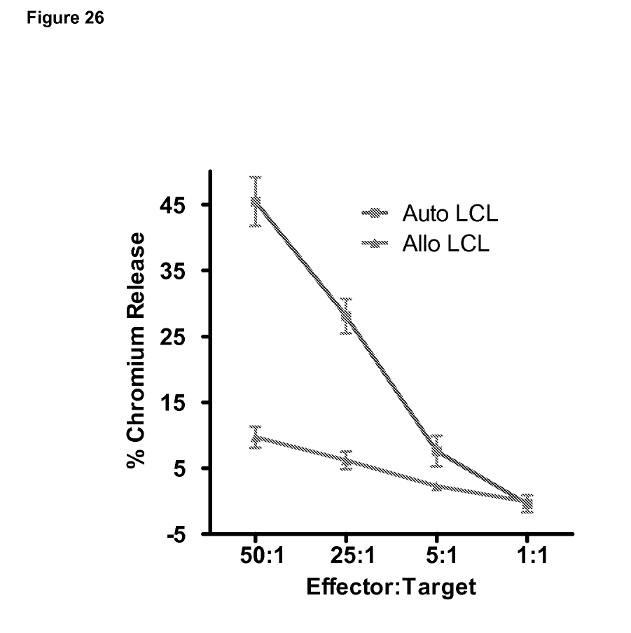

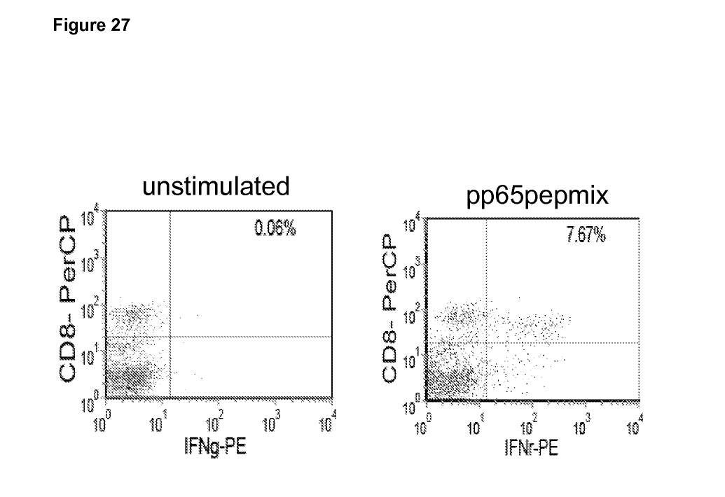

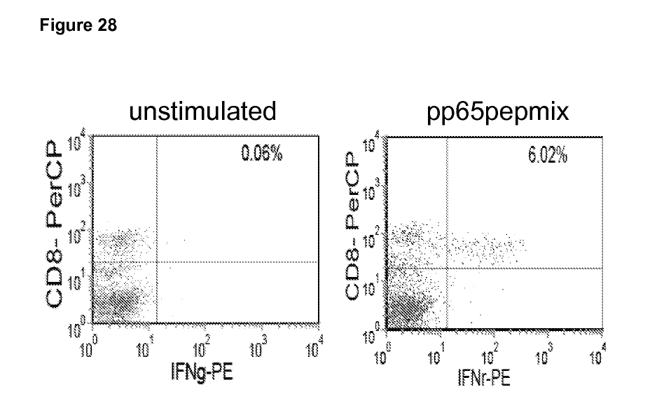

Further evidence showing successful generation of viral specific CD8+ T cells using v-APC generated with EBNA-pp65-pEK is illustrated in FIGS. 26 and 28. A 4 hour chromium release assay was performed using EBNA3C/pp65-specific T cells derived from 2nd v-APC stimulation as effectors and autologous EBV-transformed LCL (FIG. 26, top line), expressing the EBV antigen EBNA3C as Targets. LCL from HLA mismatched allogenic donor (FIG. 26, bottom line) were used as negative targets. The EBNA3C:pp65-specific T cells were able to lyse

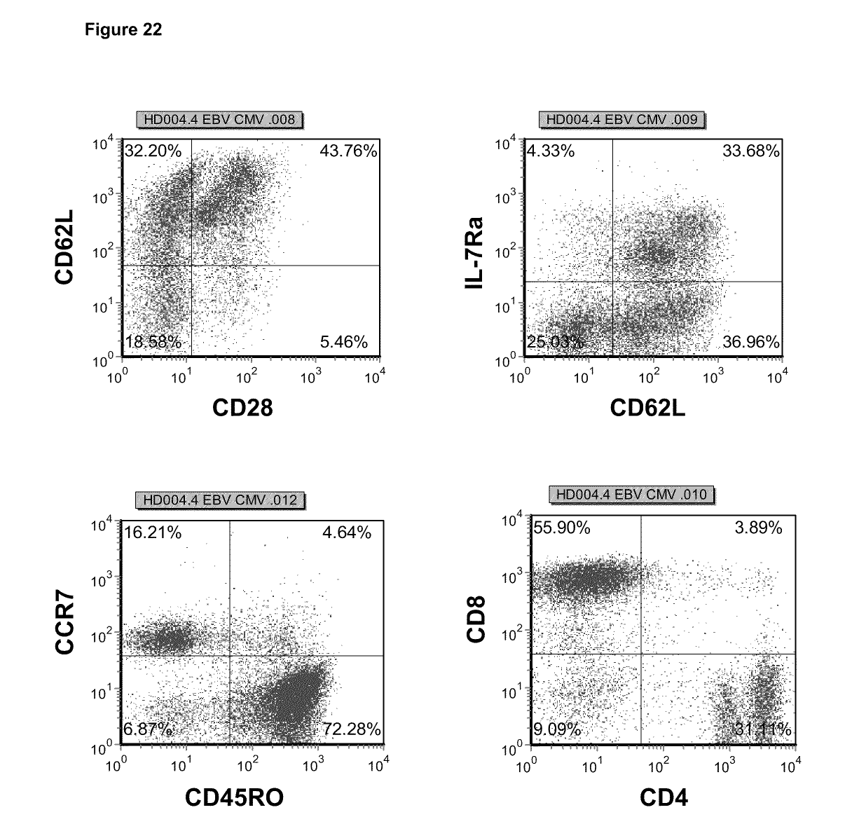

A broad response to these antigens based on multiple T cell receptor (TCR) V beta usage in resultant cultures was generated. Few of the V betas accounted for more than 10% of total V beta repertoire. A high frequency (>70%) of viral specific CD4⁺ and CD8⁺ T cells generated using this approach expressed central memory phenotypic markers such as CD45RO, CD62L, CD127, and CCR7 as illustrated in FIG. 22. Preliminary results in NOD/seid γ e^{null} mice have demonstrated that the viral specific cells generated in this system can expand in vivo in response to antigen expressing stimulators such as EBV-LCL.

PBMC modified to express viral antigen transgenes using plasmid electrotransfer can act as powerful T-APC to induce robust CMV and EBV specific T cell expansion in vitro and potentially be used as in vivo vaccines to drive expansion of these cells following adoptive transfer. This non-viral vector system for the simultaneous generation of both CMV and EBV specific T cells using intracellularly expressed viral antigen transgene fusion proteins has unique advantages in terms of in vitro and in vivo applications for facilitating anti-viral adoptive therapy.

The foregoing merely illustrates various embodiments. As such, the specific modifications discussed above are not to be construed as limitations on the scope of the disclosed products and methods. Equivalent embodiments are included within the contemplated scope. All references cited herein are incorporated by reference as if fully set forth herein.

### REFERENCES

1. Quinnan et al., New Eng. J. Med. 307:7-13, 1982;
2. Reusser et al., Blood 78:1373-1380, 1991.
3. Riddell et al., Science 257:238-241, 1992.
4. Walter et al., New Eng. J. Med. 333:1038-1044, 1995.
5. Karlsson H, Brewin J, Kinnon C, Veys P, Ammolia P J. Generation of trispecific cytotoxic T cells recognizing cytomegalovirus, adenovirus, and Epstein-Barr virus: an approach for adoptive immunotherapy of multiple pathogens. J. Immunother. 2007; 30:544-556.
6. Sun Q, Pollok K E, Burton R L, Dai L J, Britt W, Emmanuel D J, Lucas K G. Simultaneous ex vivo expansion of cytomegalovirus and Epstein-Barr virus-specific cytotoxic T lymphocytes using B lymphoblastoid cell lines expressing cytomegalovirus pp65. Blood. 1999; 94:3242-3250.
7. Meyer T, Scholz D, Warnecke G, Hunz M, Arndt R, Reischl U, Wolf H, Lissner R. Importance of simultaneous active cytomegalovirus and Epstein-Barr virus infection in renal transplantation. Clin Diagnosis Virology. 1996; 6:79-91.
8. Hamel Y, Blake N, Gabrielsson S, Haigh T, Jooss K, Martinache C, Caillat-Zucman S, Rickinson A B, Hacein-Bey S, Fischer A, Cavazzana-Calvo M. Adenovirally transduced dendritic cells induce bispecific cytotoxic T lymphocyte responses against adenovirus and cytomegalovirus pp65 or against adenovirus and Epstein-Barr virus EBNA3C protein: a novel approach for immunotherapy. Human Gene Therapy. 2002; 13:855-866.
9. Berger C, Turtle C J, Jensen M C, Riddel S R. Adoptive transfer of virus-specific and tumor-specific T cell immunity. Curr Op Immunology. 2009; 21:1-9.
10. Berger C, Jensen M C, Lansdorp P M, Gough M, Elliott C, Riddell S R. Adoptive transfer of effector CD8+ T cells derived from central memory cells establishes persistent T cell memory in primates. J Clin Invest. 2008; 118:294-305.

We claim:

1. A composition comprising a fusion multiviral chimeric antigen, wherein the fusion multiviral chimeric antigen elicits a response in a population of T cells specific to at least two separate viral antigen peptides.

2. The composition of claim 1 wherein the response includes activation a CD8+ or a CD4+ T cell.

3. The composition of claim 1 wherein the viral antigen peptides are specific to cytomegalovirus (CMV), Epstein Barr virus (EBV), Influenza A, or a combination thereof.

4. The composition of claim 3 wherein the viral antigen peptides are specific to at least three viral antigens selected from the group consisting of: CMV immediate-early protein, CMV virion envelope glycoprotein B, CMV pp65, CMV ppl 50, EBV EBNA1, EBV EBNA2, EBV EBNA3A, EBV EBNA3B, EBV EBNA3C, EBV EBNALP, EBV LMP1, EBV LMP2, and Influenza A MP1.

5. The composition of claim 1 wherein the fusion multiviral chimeric antigen has the amino acid sequence SEQ ID NO:6 or SEQ ID NO:9.

6. A vector comprising a nucleotide sequence encoding a fusion multiviral chimeric antigen, wherein the fusion multiviral chimeric antigen elicits a response in a population of T cells specific to at least two separate viral antigen peptides.

7. The vector of claim 6 wherein the fusion multiviral chimeric antigen is SEQ ID: 6 or SEQ ID: 9.

8. The vector of claim 6 wherein the nucleotide sequence is SEQ ID: 4 or SEQ ID NO:7.

9. A transduced immune cell expressing the vector of claim 6.

10. A method of obtaining an antigen presenting cell having specificity to more than one viral antigen comprising:
a) collecting peripheral blood cells from a subject;
b) transducing the cells a vector comprising a nucleotide sequence encoding a fusion multiviral chimeric antigen, wherein the fusion multiviral chimeric antigen elicits a response in a population of T cells specific to at least two separate viral antigen peptides;
c) activating the transduced cells;
c) expanding the activated cells ex vivo;
d) selecting an antigen presenting cell from the population of expanded transduced cells having multiple viral antigen specificity.

11. The method of claim 10 wherein the vector includes a TriVi construct.

12. The method of claim 10 wherein the nucleotide sequence is SEQ ID NO: 4 or SEQ ID NO: 7

13. The method of claim 10 wherein the peripheral blood cells are collected from a seronegative donor subject.

14. The method of claim 10 wherein the selected antigen presenting cell is a CD4+ or a CD8+ T cell.

15. A composition for use in adoptive immunotherapy produced by the method of claim 10.

16. A method of enhancing or reconstituting immunity in a subject suffering from or at risk of suffering from a condition selected from the group consisting of: infectious disease, autoimmune disease, graft rejection and cancer, the composition of claim 15 and a pharmaceutically acceptable carrier.

17. The method of claim 16 wherein the subject suffers is an immunodeficient or immunocompromised individual.
